(19) United States
(12) Patent Application Publication
(10) Pub. No.: US 2024/0199562 A1
(43) Pub. Date: Jun. 20, 2024

(54) UREA DERIVATIVES FOR TREATING UVEAL MELANOMA

(71) Applicants: CENTRE NATIONAL DE LA RECHERCHE SCIENTIFIQUE, PARIS (FR); INSTITUT NATIONAL DE LA SANTÉ ET DE LA RECHERCHE MÉDICALE, PARIS (FR); UNIVERSITÉ COTE D’AZUR, NICE CEDEX 2 (FR); INSTITUT CURIE, PARIS (FR)

(72) Inventors: MAEVA DUFIES, NICE (FR); GILLES PAGES, MONACO (MC); CYRIL RONCO, NICE (FR); RACHID BENHIDA, NICE (FR)

(21) Appl. No.: 18/286,569

(22) PCT Filed: Apr. 14, 2022
(86) PCT No.: PCT/EP2022/060022

§ 371 Date: Oct. 12, 2023

(30) Foreign Application Priority Data
Apr. 14, 2021 (EP) 21305489.3

(51) Int. Cl. C07D 277/82; A61K 31/4184; A61K 31/423; A61K 31/428; A61P 35/00; C07D 235/30; C07D 263/58

(52) U.S. Cl. CPC C07D 277/82; A61K 31/4184; A61K 31/423; A61K 31/428; A61P 35/00; C07D 235/30; C07D 263/58

(57) ABSTRACT

The present invention relates to ureal derivatives of formula (I) and pharmaceutical compositions comprising such derivatives for use in the treatment of uveal melanoma.

### FIELD OF THE INVENTION

The present invention relates to the field of medicine, in particular to the use of urea derivatives in the treatment of uveal melanoma.

### BACKGROUND OF THE INVENTION

Uveal melanoma is the most common intraocular malignancy tumor in adults with an incidence of about 1/100,000 new cases per year in the Western world. It may arise from any of the three parts of the uvea, and can be referred to their location, namely choroidal melanoma, ciliary body melanoma, and iris melanoma. The major proportion of uveal melanoma is represented by choroidal melanoma (85%) whereas ciliary body melanoma and iris melanoma represent 15% of uveal melanoma.

Signs and symptoms of uveal melanoma tumors when they occur can include blurred vision, double vision, reduction and also loss of vision, irritation, pain, perception of flashes or pressure in the eye. The malignant tumors can give metastases in 30 to 50% cases that may compromises patient survival.

Treatment protocols for uveal melanoma vary depending many factors such as the size of the tumor and results from testing of biopsied material from the tumor. Such treatments include removal of the affected eye (enucleation) reserved to extreme tumor burden and radiation therapies for which advances have significantly decreased the number of patients treated by enucleation in developed countries. The most common radiation treatments are proton therapy or plaque brachytherapy, in which a small disc-shaped shield (plaque) encasing radioactive seeds (using iodine-125, or ruthenium-106 and palladium-103) is attached to the outside surface of the eye, overlying the tumor. The plaque is left in place for a few days and then removed. However, the risk of metastasis after plaque radiotherapy is the same as that of enucleation, suggesting that micrometastatic spread occurs prior to treatment of the primary tumor.

Chemotherapy may represent a promising therapeutic approach for treating uveal melanoma. In previous work described in WO 2020/079184, the inventors have provided evaluated urea derivatives targeting CXCR1/CXCR2 receptors and have demonstrated that such CXCR1/CXCR2 antagonists are useful for treating cancer, such as head and neck cancer and kidney cancer, and/or disorders characterized by undesirable excessive angiogenesis, such as age-related macular degeneration.

However, uveal melanoma still remains to be investigated. There is, therefore, a need for developing drugs having a therapeutic effect against uveal melanoma.

### SUMMARY OF THE INVENTION

In this context, the inventors have surprisingly demonstrated that urea derivatives of formula (I) are useful for treating uveal melanoma, typically primary uveal melanoma and metastatic uveal melanoma. More specifically, the inventors have demonstrated a therapeutic effect for compounds of formula (I) on uveal melanoma cells derived for the primary tumor (MP38 and MP41) as well as from liver metastasis (MM66).

The present invention thus relates to a compound, a pharmaceutically acceptable salt or a tautomer thereof, of formula (I):

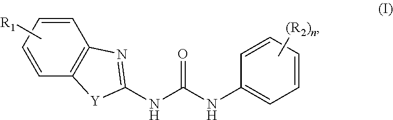

in which:
Y is —NH—, —S—, or —O—;
R₁ is a radical selected in the group consisting of:
   a hydrogen atom,
   a (C₁-C₆)alkyloxy group,
   a (C₁-C₆)alkyl group,
   a nitro group,
   a —NR₃R₄ group with R3 and R4 are independently a radical selected in the group consisting of:
      a hydrogen atom,
      a —COR₅ with R₅ is a (C₁-C₆)alkyl group, and
      a —SO₂R₆ with R₆ is a (C₁-C₆)alkyl group,
   a halogen atom, and
   a 3-14 membered ring selected in the group consisting of an aryl, a heteroaryl, a cycloalkyl, and a heterocycloalkyl, said 3-14 membered ring is optionally substituted by a radical selected in the group consisting of a (C₁-C₆)alkyl group, a (C₁-C₆)alkyloxy group, a halogen atom, a nitro group, and a carboxyl group;
R₂ is a radical selected in the group consisting of:
   a hydrogen atom,
   a halogen atom,
   a (C₁-C₆)alkyloxy group, and
   a (C₁-C₆)alkyl group;
n is an integer number from 0 to 5;
   for use for treating uveal melanoma.

In a particular embodiment, the uveal melanoma is a primary uveal melanoma or a metastatic uveal melanoma.

In a particular embodiment, Y is —S— or —O—, preferably —S—.

In a further particular embodiment, R₁ is a radical selected in the group consisting of:
a hydrogen atom,
an ethoxy group,
a methyl group,
a nitro group,
a —NR₃R₄ group with R₃ and R₄ are independently a radical selected in the group consisting of:
   a hydrogen atom,
   a —COR₅ with R₅ is a methyl group, and
   a —SO₂R₆ with R₆ is a methyl group,
   a halogen atom, preferably a bromine, and
   a 3-14 membered ring selected in the group consisting of a thiophenyl, a furanyl, a naphtalenyl, and a pyridinyl, said 3-14 membered ring is optionally substituted by a radical selected in the group consisting of a (C₁-C₆)alkyl group, a (C₁-C₆)alkyloxy group, a halogen atom, a nitro group, and a carboxyl group.

Preferably, R₁ is a radical selected in the group consisting of an ethoxy group, a nitro group, and a 3-14 membered ring selected in the group consisting of a thiophenyl, a furanyl, a naphtalenyl, and a pyridinyl, preferably a nitro group, said 3-14 membered ring is optionally substituted by a radical selected in the group consisting of a (C₁-C₆)alkyl group, a (C₁-C₆)alkyloxy group, a halogen atom, a nitro group, and a carboxyl group.

In a further particular embodiment, n is an integer number from 1 to 3, preferably 1 or 2. In a preferred embodiment, n is 1 or 2, and R₂ is in meta position.

Preferably, R₂ is a radical selected in the group consisting of:
a hydrogen atom,
a halogen atom, preferably a chlorine or a bromine, more preferably a chlorine,
a methoxy group, and
a methyl group.

In a preferred embodiment, said compound for use is of formula (IA):

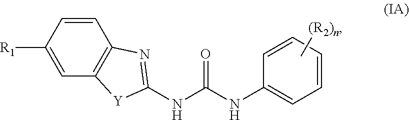

in which Y, R₁, R₂, and n are such as defined herein.

In a more preferred embodiment, said compound for use is selected in the group consisting of:
1-(1H-benzo[d]imidazol-2-yl)-3-(3-chlorophenyl)urea (MCK109);
1-(1H-benzo[d]imidazol-2-yl)-3-(2-chlorophenyl)urea (MCK110);
1-(benzo[d]oxazol-2-yl)-3-phenylurea (MCK112);
1-(1H-benzo[d]imidazol-2-yl)-3-phenylurea (MCK113);
1-(1H-benzo[d]imidazol-2-yl)-3-(4-chlorophenyl)urea (MCK115);
1-(benzo[d]thiazol-2-yl)-3-phenylurea (MCK126);
1-(benzo[d]thiazol-2-yl)-3-(2-chlorophenyl)urea (MCK127);
1-(benzo[d]thiazol-2-yl)-3-(3-chlorophenyl)urea (MCK128);
1-(benzo[d]thiazol-2-yl)-3-(4-chlorophenyl)urea (MCK129);
1-(benzo[d]thiazol-2-yl)-3-(4-methoxyphenyl)urea (MCK130);
1-(1H-benzo[d]imidazol-2-yl)-3-(4-methoxyphenyl)urea (MCK131);
1-(6-ethoxybenzo[d]thiazol-2-yl)-3-(p-tolyl)urea (MCK132);
1-(6-ethoxybenzo[d]thiazol-2-yl)-3-(o-tolyl)urea (MCK133);
1-(6-ethoxybenzo[d]thiazol-2-yl)-3-(m-tolyl)urea (MCK134);
1-(6-ethoxybenzo[d]thiazol-2-yl)-3-(4-methoxyphenyl)urea (MCK135);
1-(2-chlorophenyl)-3-(6-methylbenzo[d]thiazol-2-yl)urea (MCK136);
1-(3-chlorophenyl)-3-(6-methylbenzo[d]thiazol-2-yl)urea (MCK137);
1-(6-methylbenzo[d]thiazol-2-yl)-3-(m-tolyl)urea (MCK138);
1-(2-chlorophenyl)-3-(6-nitrobenzo[d]thiazol-2-yl)urea (MCK139);
1-(3-chlorophenyl)-3-(6-nitrobenzo[d]thiazol-2-yl)urea (MCK140);
1-(4-bromophenyl)-3-(6-nitrobenzo[d]thiazol-2-yl)urea (MCK147);
1-(6-aminobenzo[d]thiazol-2-yl)-3-(3-chlorophenyl)urea (MCK148);
N-(2-(3-(3-chlorophenyl)ureido)benzo[d]thiazol-6-yl)acetamide (MCK149);
N-(2-(3-(3-chlorophenyl)ureido)benzo[d]thiazol-6-yl)methanesulfonamide (MCK150);
1-(3,5-dichlorophenyl)-3-(6-nitrobenzo[d]thiazol-2-yl)urea (MCK151);
1-(4-methoxyphenyl)-3-(6-nitrobenzo[d]thiazol-2-yl)urea (MCK152);
1-(6-nitrobenzo[d]oxazol-2-yl)-3-(m-tolyl)urea (MCK153);
1-(5-nitrobenzo[d]oxazol-2-yl)-3-phenylurea (MCK154);
1-(6-nitro-1H-benzo[d]thiazol-2-yl)-3-phenylurea (MCK155);
1-(3-chlorophenyl)-3-(6-nitro-1H-benzo[d]imidazol-2-yl)urea (MCK156);
1-(6-nitro-1H-benzo[d]imidazol-2-yl)-3-phenylurea (MCK157);
1-(3-chlorophenyl)-3-(5-nitrobenzo[d]oxazol-2-yl)urea (MCK158);
1-(6-bromobenzo[d]thiazol-2-yl)-3-(3-chlorophenyl)urea (MCK159);
1-(3-chlorophenyl)-3-(6-nitrobenzo[d]oxazol-2-yl)urea (MCK160);
1-(3-chlorophenyl)-3-(6-(thiophen-2-yl)benzo[d]thiazol-2-yl)urea (MCK161);
1-(3-chlorophenyl)-3-(6-(furan-2-yl)benzo[d]thiazol-2-yl)urea (MCK162);
1-(3-chlorophenyl)-3-(6-(thiophen-3-yl)benzo[d]thiazol-2-yl)urea (MCK163);
1-(3-chlorophenyl)-3-(6-(naphthalen-1-yl)benzo[d]thiazol-2-yl)urea (MCK164);
1-(2-bromophenyl)-3-(6-nitrobenzo[d]thiazol-2-yl)urea (MCK165);
1-(3-chlorophenyl)-3-(6-ethoxybenzo[d]thiazol-2-yl)urea (MCK166);
1-(3,5-dichlorophenyl)-3-(6-ethoxybenzo[d]thiazol-2-yl)urea (MCK167);
1-(6-nitrobenzo[d]thiazol-2-yl)-3-(p-tolyl)urea (MCK168);
1-(4-chlorophenyl)-3-(6-nitrobenzo[d]thiazol-2-yl)urea (MCK169);
1-(3-chlorophenyl)-3-(6-(pyridin-2-yl)benzo[d]thiazol-2-yl)urea (MCK172);

1-(6-nitrobenzo[d]thiazol-2-yl)-3-(o-tolyl)urea (MCK173).

A further object of the invention is a pharmaceutical composition comprising a compound of formula (I) as defined herein, for use for treating uveal melanoma, typically primary uveal melanoma or metastatic uveal melanoma. In a particular embodiment, the pharmaceutical composition is administered by topical, oral, or parenteral route, preferably by topical or oral route.

Another object of the invention is a new compound, a pharmaceutically acceptable salt or a tautomer thereof, of formula (I):

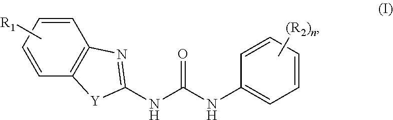

in which:
Y is —S—, or —O—;
R₁ is a radical selected in the group consisting of:
   a nitro group,
   a —NR₃R₄ group with one of R₃ or R₄ is H and the other is a —SO₂R₆ with R₆ is a (C₁-C₆)alkyl group, and
   a 3-14 membered ring selected in the group consisting of a thiophenyl, a furanyl, and a naphtalenyl, said 3-14 membered ring is optionally substituted by a radical selected in the group consisting of a (C₁-C₆)alkyl group, a (C₁-C₆)alkyloxy group, a halogen atom, a nitro group, and a carboxyl group;
      with the proviso that when Ri is a nitro group, then Y is —O—;
R₂ is a radical selected in the group consisting of:
   a hydrogen atom,
   a halogen atom,
   a (C₁-C₆)alkyloxy group, and
   a (C₁-C₆)alkyl group; and
n is an integer number from 0 to 5.

Preferably, said new compound is selected in the group consisting of:
N-(2-(3-(3-chlorophenyl)ureido)benzo[d]thiazol-6-yl)methanesulfonamide (MCK150);
1-(5-nitrobenzo[d]oxazol-2-yl)-3-phenylurea (MCK154);
1-(3-chlorophenyl)-3-(5-nitrobenzo[d]oxazol-2-yl)urea (MCK158);
1-(3-chlorophenyl)-3-(6-nitrobenzo[d]oxazol-2-yl)urea (MCK160);
1-(3-chlorophenyl)-3-(6-(thiophen-2-yl)benzo[d]thiazol-2-yl)urea (MCK161);
1-(3-chlorophenyl)-3-(6-(furan-2-yl)benzo[d]thiazol-2-yl)urea (MCK162);
1-(3-chlorophenyl)-3-(6-(thiophen-3-yl)benzo[d]thiazol-2-yl)urea (MCK163); and
1-(3-chlorophenyl)-3-(6-(naphthalen-1-yl)benzo[d]thiazol-2-yl)urea (MCK164)).

Another object of the invention is a new compound selected in the group consisting of:
N-(2-(3-(3-chlorophenyl)ureido)benzo[d]thiazol-6-yl)acetamide (MCK149);
N-(2-(3-(3-chlorophenyl)ureido)benzo[d]thiazol-6-yl)methanesulfonamide (MCK150);
1-(5-nitrobenzo[d]oxazol-2-yl)-3-phenylurea (MCK154);
1-(6-nitro-1H-benzo[d]imidazol-2-yl)-3-phenylurea (MCK157);
1-(3-chlorophenyl)-3-(5-nitrobenzo[d]oxazol-2-yl)urea (MCK158);
1-(3-chlorophenyl)-3-(6-nitrobenzo[d]oxazol-2-yl)urea (MCK160);
1-(3-chlorophenyl)-3-(6-(thiophen-2-yl)benzo[d]thiazol-2-yl)urea (MCK161);
1-(3-chlorophenyl)-3-(6-(furan-2-yl)benzo[d]thiazol-2-yl)urea (MCK162);
1-(3-chlorophenyl)-3-(6-(thiophen-3-yl)benzo[d]thiazol-2-yl)urea (MCK163);
1-(3-chlorophenyl)-3-(6-(naphthalen-1-yl)benzo[d]thiazol-2-yl)urea (MCK164);
1-(3,5-dichlorophenyl)-3-(6-ethoxybenzo[d]thiazol-2-yl)urea (MCK167); and
1-(3-chlorophenyl)-3-(6-(pyridin-2-yl)benzo[d]thiazol-2-yl)urea (MCK172).

A further object of the invention is a pharmaceutical composition comprising a new compound as defined herein, and a pharmaceutically acceptable carrier. A further object of the invention is a new compound as defined herein, for use as a medicine, preferably for use for treating a cancer.

### LEGEND OF FIGURES

FIG. 1: In vitro efficacy of MCK140 and MCK151 on uveal melanoma cells. Cells were treated for 48 hours with increasing doses of MCK140 or MCK151 and the cell viability was then evaluated by XTT.

FIG. 2A: In vitro efficacy of MCK151, Ladarixin, and AZD-5069 in uveal melanoma cells. MP41 and MM66 cells were treated for 48 hours with increasing doses of MCK151 or Ladarixin or AZD-5069 and the cell metabolism was then evaluated by XTT.

FIG. 2B: Inhibition of ROS production by MCK151 and Ladarixin in uveal melanoma cells. MP41 cells were treated for 48 hours with increasing doses of MCK151 (1 μM, 2.5 μM, and 5 μM) or Ladarixin (10 μM) and ROS quantity was evaluated by cytometry.

FIG. 3A: Evaluation of the migration ability of uveal cells treated with MCK151. MP41 and MM66 cells migration was analysed using Boyden chamber assays in the presence/absence of MCK151 (0.1 to 2.5 μM). Representative images were shown. ** p<0.01, *** p<0.001

FIG. 3B: MMP9 mRNA levels (AU) in uveal melanoma cells. MP41 and MM66 cells were treated for 24 hours with MCK151 (2.5 μM) and MMP9 mRNA levels were evaluated by qPCR. *** p<0.001.

FIG. 3C: Slug proteins amount in uveal melanoma cells treated by MCK151. MP41 and MM66 cells were treated with MCK151 (2.5 μM) for different time (0, 2, 4, 6, 8, and 24 h). SLUG protein amounts were evaluated by immunoblotting. HSP90 is used as a loading control.

FIG. 4: In vivo efficacy of MCK151 on mice treated by intraperitoneal injection. Five million MP41 cells were injected subcutaneously into the flank of 5-week-old NOD-SCID female mice. When the tumor reached 100 mm³, mice were treated every day for 20 days, by intraperitoneal injection with placebo (dextrose water vehicle) or MCK151 (200 or 400 μg in dextrose water vehicle)
A: The tumor volume was evaluated using a calliper. The results are presented as the means±sd. ** p<0.01
B: Tumor weight at the end of the experiment. ** p<0.01, *** p<0.001
C: Animal weight at the end of the experiment. ns: non significant

FIG. 5: In vivo efficacy of MCK151 on mice treated by gavage. Five million MP41 cells were injected subcutaneously into the flank of 5-week-old NOD-SCID female mice. When the tumor reached 100 mm³, mice were treated every day for 27 days, by gavage with placebo (10% ethanol vehicle) or MCK151 (33 or 100 mg/kg in 10% ethanol vehicle).
A: The tumor volume was evaluated using a calliper. The results are presented as the means±sd. ** p<0.01, *** p<0.001
B: Tumor weight at the end of the experiment. ** p<0.01, *** p<0.001 ns: non significant
C: Animal weight at the end of the experiment. ns: non significant

### DETAILED DESCRIPTION OF THE INVENTION

According to the present invention, the terms below have the following meanings:

The compounds of formula (I) include the pharmaceutically acceptable salts thereof as well as their tautomers, enantiomers, diastereoisomers, racemates of mixtures thereof, hydrates and solvates. Particularly, the compounds of formula (I) include the tautomers thereof. A tautomer of a compound of formula (I) may have the following formulae:

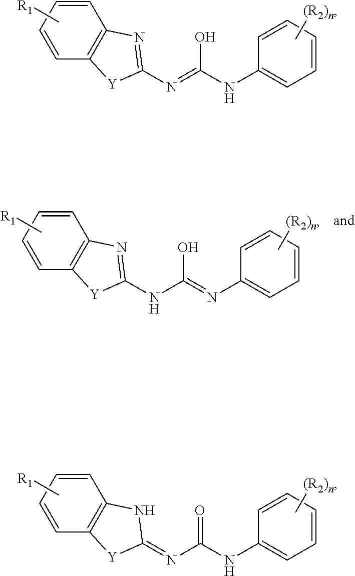

with Y, R₁, R₂, and n are such as defined herein. The terms mentioned herein with prefixes such as for example C₁-C₃ or C₁-C₆ can also be used with lower numbers of carbon atoms such as C₁-C₂, or C₁-C₅. If, for example, the term C₁-C₃ is used, it means that the corresponding hydrocarbon chain may comprise from 1 to 3 carbon atoms, especially 1, 2 or 3 carbon atoms. If, for example, the term C₁-C₆ is used, it means that the corresponding hydrocarbon chain may comprise from 1 to 6 carbon atoms, especially 1, 2, 3, 4, 5 or 6 carbon atoms.

The term “alkyl” refers to a saturated, linear or branched aliphatic group. The term “(C₁-C₃)alkyl” more specifically means methyl, ethyl, propyl, or isopropyl. The term “(C₁-C₆)alkyl” more specifically means methyl, ethyl, propyl, isopropyl, butyl, isobutyl, ter-butyl, pentyl or hexyl. In a preferred embodiment, the “alkyl” is a methyl, an ethyl, a propyl, an isopropyl, or a tert-butyl, more preferably a methyl.

The term “alkyloxy” or “alkoxy” corresponds to the alkyl group as above defined bonded to the molecule by an —O— (ether) bond. (C₁-C₃)alkyloxy includes methoxy, ethoxy, propyloxy, and isopropyloxy. (C₁-C₆)alkyloxy includes methoxy, ethoxy, propyloxy, isopropyloxy, butyloxy, isobutyloxy, tert-butyloxy, pentyloxy and hexyloxy. In a preferred embodiment, the “alkoxy” or “alkyloxy” is an ethoxy.

The term “halogen” corresponds to a fluorine, a chlorine, a bromine, or an iodine atom, preferably a chlorine or a bromine atom.

The term “3-14 membered ring” corresponds to a ring, saturated or unsaturated, having between 3 and 14 atoms, for instance 3, 4, 5, 6, 7, 8, 9, 10, 11, 12, 13, or 14 atoms. More specifically, a “3-14 membered ring” corresponds to a cycloalkyl, a heterocycloalkyl, an aryl, or a heteroaryl as defined herein.

The term “cycloalkyl” corresponds to a saturated or unsaturated mono-, bi- or tri-cyclic alkyl group comprising between 3 and 14 atoms of carbons. It also includes fused, bridged, or spiro-connected cycloalkyl groups. The term “cycloalkyl” includes for instance cyclopropyl, cyclobutyl, cyclopentyl, and cyclohexyl.

The term “heterocycloalkyl” corresponds to a saturated or unsaturated cycloalkyl group as above defined further comprising at least one heteroatom such as nitrogen, oxygen, or sulphur atom, preferably at least one nitrogen atom. It also includes fused, bridged, or spiro-connected heterocycloalkyl groups. Representative heterocycloalkyl groups include, but are not limited to dioxolanyl, benzo[1,3]dioxolyl, azetidinyl, oxetanyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, imidazolidinyl, morpholinyl, 1,4-dithianyl, pyrrolidinyl, oxozolinyl, oxazolidinyl, isoxazolinyl, isoxazolidinyl, thiazolinyl, thiazolidinyl, isothiazolinyl, isothiazolidinyl, dihydropyranyl, tetrahydropyranyl, tetrahydrofuranyl, and tetrahydrothiophenyl.

The term “aryl” corresponds to a mono- or bi-cyclic aromatic hydrocarbons having from 6 to 12 carbon atoms. For instance, the term “aryl” includes phenyl, naphthyl or naphtalenyl, or anthracenyl. In a preferred embodiment, the aryl is a naphtalenyl. The term “heteroaryl” as used herein corresponds to an aromatic, mono- or poly-cyclic group comprising between 5 and 14 atoms and comprising at least one heteroatom such as nitrogen, oxygen or sulphur atom. As used herein, the term “heteroaryl” further includes the “fused arylheterocycloalkyl” and “fused heteroarylcycloalkyl”. The terms “fused arylheterocycloalkyl” and “fused heteroarylcycloalkyl” correspond to a bicyclic group in which an aryl as above defined or a heteroaryl is respectively bounded to the heterocycloalkyl or the cycloalkyl as above defined by at least two carbons. In other terms, the aryl or the heteroaryl shares a carbon bond with the heterocycloalkyl or the cycloalkyl. Examples of such mono- and poly-cyclic heteroaryl group, fused arylheterocycloalkyl and fused arylcycloalkyl may be: pyridinyl, thiazolyl, thiophenyl, furanyl, pyrrolyl, pyrazolyl, imidazolyl, triazolyl, tetrazolyl, benzofuranyl, thianaphthalenyl, indolyl, indolinyl, quinolinyl, isoquinolinyl, benzimidazolyl, tetrahydroquinolinyl, tetrahydroisoquinolinyl, triazinyl, thianthrenyl, isobenzofuranyl, chromenyl, xanthenyl, phenoxanthinyl, isothiazolyl, isoxazolyl, pyrazinyl, pyridazinyl, indolizinyl, isoindolyl, indazolyl, purinyl, quinolizinyl, phtalazinyl, naphthyridinyl, quinoxalinyl, quinazolinyl, cinnolinyl, pteridinyl, carbazolyl, ß-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, indolinyl, isoindolinyl, oxazolidinyl, benzotriazolyl, benzoisoxazolyl, oxindolyl, benzoxazolyl, benzoxazolinyl, benzoxazinyl, benzothienyl, benzothiazolyl, benzodiazepinyl, benzazepinyl, benzoxazepinyl, isatinyl, dihydropyridyl, pyrimidinyl, s-triazinyl, oxazolyl, or thiofuranyl. In a preferred embodiment, the heteroaryl is a thiophenyl, a furanyl or a pyridinyl.

The expression “substituted by” means that the group or radical is substituted by one or several radicals of the list. The expression “optionally substituted” means that the group or radical is not substituted or substituted by one or several radicals of the list.

As used herein, the term “pharmaceutically acceptable salt” includes inorganic as well as organic acids salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically acceptable inorganic or organic acid addition salts include the pharmaceutically acceptable salts listed in J. Pharm. Sci. 1977, 66, 2, and in Handbook of Pharmaceutical Salts: Properties, Selection, and Use edited by P. Heinrich Stahl and Camille G. Wermuth 2002. In a preferred embodiment, the salt is a hydrochloride salt.

### Compounds

An object of the invention is a compound, a pharmaceutically acceptable salt or a tautomer thereof, of formula (I) for use for treating uveal melanoma, typically primary uveal melanoma or metastatic uveal melanoma.

The present invention thus relates to a compound, a pharmaceutically acceptable salt or a tautomer thereof, of formula (I):

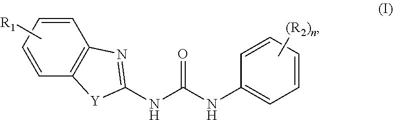

in which:
Y is —NH—, —S—, or —O—;
R₁ is a radical selected in the group consisting of:
   a hydrogen atom,
   a (C₁-C₆)alkyloxy group,
   a (C₁-C₆)alkyl group,
   a nitro group,
   a —NR₃R₄ group with R₃ and R₄ are independently a radical selected in the group consisting of:
      a hydrogen atom,
      a —COR₅ with R₅ is a (C₁-C₆)alkyl group, and
      a —SO₂R₆ with R₆ is a (C₁-C₆)alkyl group,
   a halogen atom, and
   a 3-14 membered ring selected in the group consisting of an aryl, a heteroaryl, a cycloalkyl, and a heterocycloalkyl, said 3-14 membered ring is optionally substituted by a radical selected in the group consisting of a (C₁-C₆)alkyl group, a (C₁-C₆)alkyloxy group, a halogen atom, a nitro group, and a carboxyl group;
R₂ is a radical selected in the group consisting of:
   a hydrogen atom,
   a halogen atom,
   a (C₁-C₆)alkyloxy group, and
   a (C₁-C₆)alkyl group;
n is an integer number from 0 to 5;
   for use for treating uveal melanoma.

According to the invention, a compound for use of formula (I) is such that Y is —NH—, —S—, or —O—. In a particular embodiment, Y is —S—, or —O—. In a preferred embodiment, Y is —S.

According to the invention, a compound for use of formula (I) is such that R₁ is a radical selected in the group consisting of:
a hydrogen atom,
a (C₁-C₆)alkyloxy group,
a (C₁-C₆)alkyl group,
a nitro group,
a —NR₃R₄ group with R₃ and R₄ are independently a radical selected in the group consisting of:
   a hydrogen atom,
   a —COR₅ with R₅ is a (C₁-C₆)alkyl group, and
   a —SO₂R₆ with R6 is a (C₁-C₆)alkyl group,
a halogen atom, and
a 3-14 membered ring selected in the group consisting of an aryl, a heteroaryl, a cycloalkyl, and a heterocycloalkyl, said 3-14 membered ring is optionally substituted by a radical selected in the group consisting of a (C₁-C₆)alkyl group, a (C₁-C₆)alkyloxy group, a halogen atom, a nitro group, and a carboxyl group.

In a particular embodiment, R₁ is a hydrogen atom.

In a particular embodiment, R₁ is a (C₁-C₆)alkyloxy group. Preferably, R₁ is an ethoxy group.

In a particular embodiment, R₁ is a (C₁-C₆)alkyl group. Preferably, R₁ is a methyl group.

In a particular embodiment, R₁ is a nitro group.

In a particular embodiment, R₁ is a —NR₃R₄ group with R₃ and R₄ are independently a radical selected in the group consisting of:
a hydrogen atom,
a —COR₅with R₅ is a (C₁-C₆)alkyl group, and
a —SO₂R₆ with R₆ is a (C₁-C₆)alkyl group.

Preferably, R₁ is a —NR₃R₄ group with R₃ and R₄ are independently a radical selected in the group consisting of:
a hydrogen atom,
a —COR₅ with R₅ is a methyl group, and
a —SO₂R₆ with R₆ is a methyl group.

More preferably, R₁ is —NH₂, —NH—CO—CH₃, or —NH—SO₂—CH₃.

In a particular embodiment, R₁ is a halogen atom. Preferably, R₁ is a bromine.

In a particular embodiment, R₁ is a 3-14 membered ring selected in the group consisting of an aryl, a heteroaryl, a cycloalkyl, and a heterocycloalkyl, said 3-14 membered ring is optionally substituted by a radical selected in the group consisting of a (C₁-C₆)alkyl group, a (C₁-C₆)alkyloxy group, a halogen atom, a nitro group, and a carboxyl group. In a preferred embodiment, R₁ is a 3-14 membered ring selected in the group consisting of an aryl and a heteroaryl optionally substituted by a radical selected in the group consisting of a (C₁-C₆)alkyl group, a (C₁-C₆)alkyloxy group, a halogen atom, a nitro group, and a carboxyl group. In a more preferred embodiment, R₁ is a 3-14 membered ring selected in the group consisting of a thiophenyl, a furanyl, a naphtalenyl, and a pyridinyl, said 3-14 membered ring is optionally substituted by a radical selected in the group consisting of a (C₁-C₆)alkyl group, a (C₁-C₆)alkyloxy group, a halogen atom, a nitro group, and a carboxyl group. In an even more preferred embodiment, R₁ is a thiophenyl, a furanyl, a naphtalenyl, or a pyridinyl.

According to a preferred embodiment of the invention, a compound for use of formula (I) is such that R₁ is a radical selected in the group consisting of:
a hydrogen atom,
an ethoxy group,
a methyl group,
a nitro group,
a —NR₃R₄ group with R₃ and R₄ are independently a radical selected in the group consisting of:
   a hydrogen atom,
   a —COR₅ with R₅ is a methyl group, and
   a —SO₂R₆ with R₆ is a methyl group,
a halogen atom, preferably a bromine, and
a 3-14 membered ring selected in the group consisting of a thiophenyl, a furanyl, a naphtalenyl, and a pyridinyl , said 3-14 membered ring is optionally substituted by a radical selected in the group consisting of a (C₁-C₆)alkyl group, a (C₁-C₆)alkyloxy group, a halogen atom, a nitro group, and a carboxyl group.

According to a more preferred embodiment of the invention, a compound for use of formula (I) is such that R₁ is a radical selected in the group consisting of an ethoxy group, a nitro group, and a 3-14 membered ring selected in the group consisting of a thiophenyl, a furanyl, a naphtalenyl, and a pyridinyl, preferably a nitro group, said 3-14 membered ring is optionally substituted by a radical selected in the group consisting of a (C₁-C₆)alkyl group, a (C₁-C₆)alkyloxy group, a halogen atom, a nitro group, and a carboxyl group.

As defined herein, a compound for use of formula (I) is substituted by a —(R₂)ₙ group on the phenyl. A —(R₂)ₙ group correspond to n radicals R₂ substituted in the phenyl. According to the invention, a compound for use of formula (I) is such that n is an integer number from 0 to 5. For instance, if n is 0, then the phenyl is unsubstituted. If n is 1, then the phenyl is monosubstituted by a radical R₂ or substituted by only one radical R₂. If n is 2, then the phenyl is disubstituted by a radical R₂ or substituted by two radicals R₂. If n is 3, then the phenyl is trisubstituted by a radical R₂ or substituted by three radicals R₂. If n is 4, then the phenyl is tetrasubstituted by a radical R₂ or substituted by four radicals R₂. If n is 5, then the phenyl is pentasubstituted by a radical R₂ or substituted by five radicals R₂. It is well understood that the groups R₂, when n is from 2 and 5 may be identical or different.

According to the invention, a compound for use of formula (I) is such that n is an integer number from 0 to 5. Preferably, n is an integer number from 1 to 3. More preferably, n is an integer number of 1 or 2.

According to the invention, a compound for use of formula (I) is such that R₂ is a radical selected in the group consisting of:
a hydrogen atom,
a halogen atom,
a (C₁-C₆)alkyloxy group, and
a (C₁-C₆)alkyl group.

According to a preferred embodiment, a compound for use of formula (I) is such that R₂ is a radical selected in the group consisting of:
a hydrogen atom,
a halogen atom, preferably a chlorine or a bromine, more preferably a chlorine,
a methoxy group, and
a methyl group.

In a particular embodiment, a compound for use of formula (I) is such that n is 1 or 2 and R₂ is in meta position.

In a more particular embodiment, a compound for use of formula (I) is such that n is 1 and R₂ is a radical as above defined in meta position. According to this particular embodiment, such a compound for use is of the following formula:

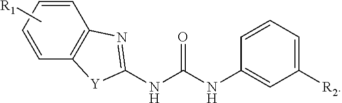

In a further more particular embodiment, a compound for use of formula (I) is such that n is 2 and R₂ is a radical as above defined in meta position. According to this particular embodiment, such a compound for use is of the following formula:

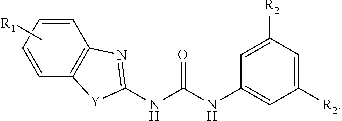

In a particular embodiment, a compound for use according to the invention is such that said compound is of formula (IA):

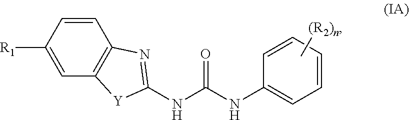

in which Y, R₁, R₂, and n are such as defined including all the particular and preferred embodiments.

In a further particular embodiment, a compound for use according to the invention is such that said compound is of formula (IB):

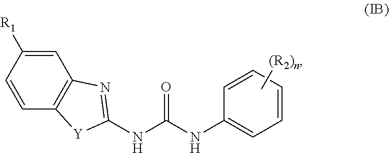

in which Y, R₁, R₂, and n are such as defined including all the particular and preferred embodiments.

A preferred embodiment of the invention is a compound of formula (I), preferably of formula (IA) for use for treating uveal melanoma, in which:
Y is —S—;
R₁ is a nitro group;
R₂ is a halogen, preferably a chlorine; and
n is 1 or 2.

In a more preferred embodiment, a compound of formula (I) for use for treating uveal melanoma is selected in the group consisting of:
1-(1H-benzo[d]imidazol-2-yl)-3-(3-chlorophenyl)urea (MCK109);
1-(1H-benzo[d]imidazol-2-yl)-3-(2-chlorophenyl)urea (MCK110);
1-(benzo[d]oxazol-2-yl)-3-phenylurea (MCK112);
1-(1H-benzo[d]imidazol-2-yl)-3-phenylurea (MCK113);
1-(1H-benzo[d]imidazol-2-yl)-3-(4-chlorophenyl)urea (MCK115);
1-(benzo[d]thiazol-2-yl)-3-phenylurea (MCK126);
1-(benzo[d]thiazol-2-yl)-3-(2-chlorophenyl)urea (MCK127);
1-(benzo[d]thiazol-2-yl)-3-(3-chlorophenyl)urea (MCK128);
1-(benzo[d]thiazol-2-yl)-3-(4-chlorophenyl)urea (MCK129);
1-(benzo[d]thiazol-2-yl)-3-(4-methoxyphenyl)urea (MCK130);
1-(1H-benzo[d]imidazol-2-yl)-3-(4-methoxyphenyl)urea (MCK131);
1-(6-ethoxybenzo[d]thiazol-2-yl)-3-(p-tolyl)urea (MCK132);
1-(6-ethoxybenzo[d]thiazol-2-yl)-3-(o-tolyl)urea (MCK133);
1-(6-ethoxybenzo[d]thiazol-2-yl)-3-(m-tolyl)urea (MCK134);
1-(6-ethoxybenzo[d]thiazol-2-yl)-3-(4-methoxyphenyl)urea (MCK135);
1-(2-chlorophenyl)-3-(6-methylbenzo[d]thiazol-2-yl)urea (MCK136);
1-(3-chlorophenyl)-3-(6-methylbenzo[d]thiazol-2-yl)urea (MCK137);
1-(6-methylbenzo[d]thiazol-2-yl)-3-(m-tolyl)urea (MCK138);
1-(2-chlorophenyl)-3-(6-nitrobenzo[d]thiazol-2-yl)urea (MCK139);
1-(3-chlorophenyl)-3-(6-nitrobenzo[d]thiazol-2-yl)urea (MCK140);
1-(4-bromophenyl)-3-(6-nitrobenzo[d]thiazol-2-yl)urea (MCK147);
1-(6-aminobenzo[d]thiazol-2-yl)-3-(3-chlorophenyl)urea (MCK148);
N-(2-(3-(3-chlorophenyl)ureido)benzo[d]thiazol-6-yl)acetamide (MCK149);
N-(2-(3-(3-chlorophenyl)ureido)benzo[d]thiazol-6-yl)methanesulfonamide (MCK150);
1-(3,5-dichlorophenyl)-3-(6-nitrobenzo[d]thiazol-2-yl)urea (MCK151);
1-(4-methoxyphenyl)-3-(6-nitrobenzo[d]thiazol-2-yl)urea (MCK152);
1-(6-nitrobenzo[d]oxazol-2-yl)-3-(m-tolyl)urea (MCK153);
1-(5-nitrobenzo[d]oxazol-2-yl)-3-phenylurea (MCK154);
1-(6-nitro-1H-benzo[d]thiazol-2-yl)-3-phenylurea (MCK155);
1-(3-chlorophenyl)-3-(6-nitro-1H-benzo[d]imidazol-2-yl)urea (MCK156);
1-(6-nitro-1H-benzo[d]imidazol-2-yl)-3-phenylurea (MCK157);
1-(3-chlorophenyl)-3-(5-nitrobenzo[d]oxazol-2-yl)urea (MCK158);
1-(6-bromobenzo[d]thiazol-2-yl)-3-(3-chlorophenyl)urea (MCK159);
1-(3-chlorophenyl)-3-(6-nitrobenzo[d]oxazol-2-yl)urea (MCK160);
1-(3-chlorophenyl)-3-(6-(thiophen-2-yl)benzo[d]thiazol-2-yl)urea (MCK161);
1-(3-chlorophenyl)-3-(6-(furan-2-yl)benzo[d]thiazol-2-yl)urea (MCK162);
1-(3-chlorophenyl)-3-(6-(thiophen-3-yl)benzo[d]thiazol-2-yl)urea (MCK163);
1-(3-chlorophenyl)-3-(6-(naphthalen-1-yl)benzo[d]thiazol-2-yl)urea (MCK164);
1-(2-bromophenyl)-3-(6-nitrobenzo[d]thiazol-2-yl)urea (MCK165);
1-(3-chlorophenyl)-3-(6-ethoxybenzo[d]thiazol-2-yl)urea (MCK166);
1-(3,5-dichlorophenyl)-3-(6-ethoxybenzo[d]thiazol-2-yl)urea (MCK167);
1-(6-nitrobenzo[d]thiazol-2-yl)-3-(p-tolyl)urea (MCK168);
1-(4-chlorophenyl)-3-(6-nitrobenzo[d]thiazol-2-yl)urea (MCK169);
1-(3-chlorophenyl)-3-(6-(pyridin-2-yl)benzo[d]thiazol-2-yl)urea (MCK172);
1-(6-nitrobenzo[d]thiazol-2-yl)-3-(o-tolyl)urea (MCK173).

A further object of the invention is a new compound, a pharmaceutically acceptable salt or a tautomer thereof, of formula (I):

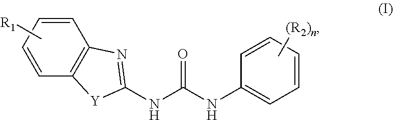

in which:
Y is —S—, or —O—;
R₁ is a radical selected in the group consisting of:
   a nitro group,
   a —NR₃R₄ group with one of R₃ or R₄ is H and the other is a radical selected in the group consisting of:
      a —COR₅ with R₅ is a (C₁-C₆)alkyl group, and
      a —SO₂R₆ with R₆ is a (C₁-C₆)alkyl group, preferably a —SO₂R₆ with R₆ is a (C₁-C₆)alkyl group, and
   a 3-14 membered ring selected in the group consisting of an aryl, a heteroaryl, a cycloalkyl, and a heterocycloalkyl, preferably selected in the group consisting of a thiophenyl, a furanyl, a naphtalenyl, and a pyridinyl, said 3-14 membered ring is optionally substituted by a radical selected in the group consisting of a (C₁-C₆)alkyl group, a (C₁-C₆)alkyloxy group, a halogen atom, a nitro group, and a carboxyl group;
      with the proviso that when R₁ is a nitro group, then Y is —O—;
R₂ is a radical selected in the group consisting of:
   a hydrogen atom,
   a halogen atom,
   a (C₁-C₆)alkyloxy group, and
   a (C₁-C₆)alkyl group; and
n is an integer number from 0 to 5.

In a particular embodiment, a new compound of formula (I) is such that:
Y is —S—;
R₁ is a radical selected in the group consisting of:
   a —NR₃R₄ group with one of R₃ or R₄ is H and the other is a —SO₂R₆ with R₆ is a (C₁-C₆)alkyl group, and
   a 3-14 membered ring selected in the group consisting of an aryl, a heteroaryl, a cycloalkyl, and a heterocycloalkyl, preferably selected in the group consisting of a thiophenyl, a furanyl, a naphtalenyl, and a pyridinyl, said 3-14 membered ring is optionally substituted by a radical selected in the group consisting of a (C₁-C₆)alkyl group, a (C₁-C₆)alkyloxy group, a halogen atom, a nitro group, and a carboxyl group;
R₂ is a radical selected in the group consisting of:
   a hydrogen atom,
   a halogen atom,
   a (C₁-C₆)alkyloxy group, and
   a (C₁-C₆)alkyl group; and
n is an integer number from 0 to 5, preferably n is 1 or 2, more preferably n is 1.

In a further particular embodiment, a new compound of formula (I) is such that:
Y is —O—;
R₁ is a radical selected in the group consisting of:
   a nitro group,
   a —NR₃R₄ group with one of R₃ or R₄ is H and the other is a radical selected in the group consisting of:
      a —COR₅ with R₅ is a (C₁-C₆)alkyl group, and
      a —SO₂R₆ with R₆ is a (C₁-C₆)alkyl group, and
   a 3-14 membered ring selected in the group consisting of an aryl, a heteroaryl, a cycloalkyl, and a heterocycloalkyl, preferably selected in the group consisting of a thiophenyl, a furanyl, a naphtalenyl, and a pyridinyl, said 3-14 membered ring is optionally substituted by a radical selected in the group consisting of a (C₁-C₆)alkyl group, a (C₁-C₆)alkyloxy group, a halogen atom, a nitro group, and a carboxyl group;
R₂ is a radical selected in the group consisting of:
   a hydrogen atom,
   a halogen atom,
   a (C₁-C₆)alkyloxy group, and
   a (C₁-C₆)alkyl group; and
n is an integer number from 0 to 5, preferably n is 1 or 2, more preferably n is 1.

In a preferred embodiment, a new compound of formula (I) is such that:
Y is —O—;
R₁ is a radical selected in the group consisting of:
   a nitro group,
   a 3-14 membered ring selected in the group consisting of an aryl, a heteroaryl, a cycloalkyl, and a heterocycloalkyl, preferably selected in the group consisting of a thiophenyl, a furanyl, a naphtalenyl, and a pyridinyl, said 3-14 membered ring is optionally substituted by a radical selected in the group consisting of a (C₁-C₆)alkyl group, a (C₁-C₆)alkyloxy group, a halogen atom, a nitro group, and a carboxyl group;
R₂ is a radical selected in the group consisting of:
   a hydrogen atom,
   a halogen atom,
   a (C₁-C₆)alkyloxy group, and
   a (C₁-C₆)alkyl group; and
n is an integer number from 0 to 5, preferably n is 1 or 2, more preferably n is 1.

A preferred new compound of formula (I) is selected in the group consisting of:
N-(2-(3-(3-chlorophenyl)ureido)benzo[d]thiazol-6-yl)methanesulfonamide (MCK150);
1-(5-nitrobenzo[d]oxazol-2-yl)-3-phenylurea (MCK154);
1-(3-chlorophenyl)-3-(5-nitrobenzo[d]oxazol-2-yl)urea (MCK158);
1-(3-chlorophenyl)-3-(6-nitrobenzo[d]oxazol-2-yl)urea (MCK160);
1-(3-chlorophenyl)-3-(6-(thiophen-2-yl)benzo[d]thiazol-2-yl)urea (MCK161);
1-(3-chlorophenyl)-3-(6-(furan-2-yl)benzo[d]thiazol-2-yl)urea (MCK162);
1-(3-chlorophenyl)-3-(6-(thiophen-3-yl)benzo[d]thiazol-2-yl)urea (MCK163); and
1-(3-chlorophenyl)-3-(6-(naphthalen-1-yl)benzo[d]thiazol-2-yl)urea (MCK164).

A further object of the invention is a new compound selected in the group consisting of:
N-(2-(3-(3-chlorophenyl)ureido)benzo[d]thiazol-6-yl)acetamide (MCK149);

N-(2-(3-(3-chlorophenyl)ureido)benzo[d]thiazol-6-yl)methanesulfonamide (MCK150);
1-(5-nitrobenzo[d]oxazol-2-yl)-3-phenylurea (MCK154);
1-(6-nitro-1H-benzo[d]imidazol-2-yl)-3-phenylurea (MCK157);
1-(3-chlorophenyl)-3-(5-nitrobenzo[d]oxazol-2-yl)urea (MCK158);
1-(3-chlorophenyl)-3-(6-nitrobenzo[d]oxazol-2-yl)urea (MCK160);
1-(3-chlorophenyl)-3-(6-(thiophen-2-yl)benzo[d]thiazol-2-yl)urea (MCK161);
1-(3-chlorophenyl)-3-(6-(furan-2-yl)benzo[d]thiazol-2-yl)urea (MCK162);
1-(3-chlorophenyl)-3-(6-(thiophen-3-yl)benzo[d]thiazol-2-yl)urea (MCK163);
1-(3-chlorophenyl)-3-(6-(naphthalen-1-yl)benzo[d]thiazol-2-yl)urea (MCK164);
1-(3,5-dichlorophenyl)-3-(6-ethoxybenzo[d]thiazol-2-yl)urea (MCK167); and
1-(3-chlorophenyl)-3-(6-(pyridin-2-yl)benzo[d]thiazol-2-yl)urea (MCK172).

### Application

According to the present invention, the terms below have the following meanings:

As used herein, the terms “treatment”, “treat” or “treating” refer to any act intended to ameliorate the health status of patients such as therapy, prevention, prophylaxis and retardation of a disease, particularly uveal melanoma. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to an animal, preferably to a mammal, even more preferably to a human.

The terms “quantity,” “amount,” and “dose” are used interchangeably herein and may refer to an absolute quantification of a molecule.

As used herein, the terms “active principle”, “active ingredient” and “active pharmaceutical ingredient” are equivalent and refer to a component of a pharmaceutical composition having a therapeutic effect. Particularly, such terms designate a compound of formula (I), (IA) or (IB).

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder, particularly uveal melanoma, such as primary uveal melanoma or metastatic uveal melanoma or any other cancer.

As used herein, the term “effective amount” refers to a quantity of an active ingredient or of a pharmaceutical composition that prevents, removes or reduces the deleterious effects of the disease, particularly uveal melanoma, such as primary uveal melanoma or metastatic uveal melanoma or any other cancer. It is obvious that the quantity to be administered can be adapted by the man skilled in the art according to the subject to be treated, to the nature of the disease, etc. In particular, doses and regimen of administration may be adapted to the nature, the stage and the severity of the disease to be treated, as well as the weight, the age and the global health of the subject to be treated, as well as the judgment of the doctor.

As used herein, the term “excipient or pharmaceutically acceptable carrier” refers to any ingredient except active ingredients that is present in a pharmaceutical composition. Its addition may be aimed to confer a particular consistency or other physical or gustative properties to the final product. An excipient or pharmaceutically acceptable carrier must be devoid of any interaction, in particular chemical, with the active ingredients.

The present invention relates to a compound of formula (I) as defined herein, a pharmaceutically acceptable salt or a tautomer thereof, for use for treating uveal melanoma, typically primary uveal melanoma or metastatic uveal melanoma.

The present invention further relates to a method for treating uveal melanoma, typically primary uveal melanoma or metastatic uveal melanoma, comprising administering in a subject in need thereof an effective amount of a compound of formula (I) as defined herein, a pharmaceutically acceptable salt or a tautomer thereof.

The present invention also relates to a use of a compound of formula (I) as defined herein, a pharmaceutically acceptable salt or a tautomer thereof, for the manufacture of a drug or a medicament, for treating uveal melanoma, typically primary uveal melanoma or metastatic uveal melanoma.

The present invention further relates to a pharmaceutical composition comprising a compound of formula (I) as defined herein, a pharmaceutically acceptable salt or a tautomer thereof, for use for treating uveal melanoma, typically primary uveal melanoma or metastatic uveal melanoma.

The present invention further relates to a method for treating uveal melanoma, typically primary uveal melanoma or metastatic uveal melanoma, comprising administering in a subject in need thereof an effective amount of a pharmaceutical composition comprising a compound of formula (I) as defined herein, a pharmaceutically acceptable salt or a tautomer thereof. The present invention also relates to a use of a compound of formula (I) as defined herein, a pharmaceutically acceptable salt or a tautomer thereof, for the manufacture of a drug, a medicament, or a pharmaceutical composition for treating uveal melanoma, typically primary uveal melanoma or metastatic uveal melanoma.

In a particular embodiment, the pharmaceutical composition for use as defined herein comprises a compound of formula (I) in a dose from 1 to 1000 mg/kg BW, preferably from 10 to 250 mg/kg BW, more preferably from 50 to 200 mg/kg BW. An object of the invention is thus a pharmaceutical composition for use as disclosed herein, in which said composition is administered at a dose from 1 to 1000 mg/kg BW, preferably from 10 to 250 mg/kg BW, more preferably from 50 to 200 mg/kg BW. As used herein, the term “BW” means bodyweight.

In a particular aspect, the compounds and the pharmaceutical compositions for use of the invention can be administered 4, 5, 6 or 7 days a week during 1, 2, 3, 4, 5, 6 or 7 weeks. Optionally, several treatment cycles can be performed, optionally with a break period between two treatment cycles, for instance of 1, 2, 3, 4 or 5 weeks.

The administration route can be topical, transdermal, oral, rectal, sublingual, intranasal, intrathecal, intratumoral or parenteral (including subcutaneous, intramuscular, intraperitoneal, intravenous and/or intradermal). Preferably, the administration route is topical, oral or parenteral. More preferably, the administration route is topical or oral. The pharmaceutical composition is adapted for one or several of the above-mentioned routes.

The pharmaceutical composition can be formulated as solutions in pharmaceutically compatible solvents or as emulsions, suspensions or dispersions in suitable pharmaceutical solvents or vehicles, or as pills, tablets or capsules that contain solid vehicles in a way known in the art. Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. Formulations for rectal administration may be in the form of a suppository incorporating the active ingredient and carrier such as cocoa butter, or in the form of an enema. Formulations suitable for parenteral administration conveniently comprise a sterile oily or aqueous preparation of the active ingredient which is preferably isotonic with the blood of the recipient. Formulations for topical application may be in the form of cream, lotion, ointment, in the form of an oil-in-water emulsion or a water-in-oil emulsion. Every such formulation can also contain other pharmaceutically compatible and nontoxic auxiliary agents, such as stabilizers, antioxidants, binders, dyes, emulsifiers or flavoring substances. The formulations of the present invention comprise an active ingredient in association with a pharmaceutically acceptable carrier, and optionally other therapeutic ingredients. The carrier must be “acceptable” in the sense of being compatible with the other ingredients of the formulations and not deleterious to the recipient thereof. Methods for the safe and effective administration of most of these chemotherapeutic agents are known to those skilled in the art. In addition, their administration is described in the standard literature.

Another object of the invention is a pharmaceutical composition comprising a new compound of formula (I) as defined herein, and a pharmaceutically acceptable carrier. A further object of the invention is a new compound of formula (I) as defined herein for use as a drug or a medicine.

A further object of the invention is a pharmaceutical composition comprising a new compound of formula (I) as defined herein, and a pharmaceutically acceptable carrier for use for treating a cancer. The present invention further relates to a method for treating a cancer comprising administering in a subject in need thereof an effective amount of a new compound of formula (I) as defined herein, a pharmaceutically acceptable salt or a tautomer thereof. The present invention also relates to a use of a new compound of formula (I) as defined herein, a pharmaceutically acceptable salt or a tautomer thereof, for the manufacture of a drug, a medicament, or a pharmaceutical composition for treating a cancer.

The cancer can be a solid tumor or a hematopoietic cancer. For instance, the cancer can be selected from the group consisting of bone cancer, gastrointestinal cancer, liver cancer, pancreatic cancer, gastric cancer, colorectal cancer, esophageal cancer, oro-pharyngeal cancer, laryngeal cancer, salivary gland carcinoma, thyroid cancer, lung cancer, cancer of the head or neck, skin cancer, squamous cell cancer, melanoma, uterine cancer, cervical cancer, endometrial carcinoma, vulvar cancer, ovarian cancer, breast cancer, prostate cancer, cancer of the endocrine system, sarcoma of soft tissue, bladder cancer, kidney cancer, glioblastoma, and various types of cancers of the central nervous system, lymphoma and leukemia. Preferably the cancer is leukemia, kidney cancer, medulloblastoma, head and neck cancer, and triple-negative breast cancer.

A further object of the invention is a new compound of formula (I) as defined herein or a pharmaceutical composition comprising a new compound of formula (I) as defined herein, and a pharmaceutically acceptable carrier for use for treating an ocular disease associated with angiogenesis. The present invention further relates to a method for treating an ocular disease associated with angiogenesis comprising administering in a subject in need thereof an effective amount of a new compound of formula (I) as defined herein, a pharmaceutically acceptable salt or a tautomer thereof. The present invention also relates to a use of a new compound of formula (I) as defined herein, a pharmaceutically acceptable salt or a tautomer thereof, for the manufacture of a drug, a medicament, or a pharmaceutical composition for treating an ocular disease associated with angiogenesis. Ocular disease associated with angiogenesis includes corneal graft angiogenesis, neovascular glaucoma, diabetic retinopathy, corneal diseases induced by new blood vessels, macular degeneration, pterygium, retinal degeneration, retrolental fibroplasia, granular conjunctivitis, and the like

Further aspects and advantages of the present invention will be described in the following examples, which should be regarded as illustrative and not limiting.

### EXAMPLES

### Example A: Chemistry

The compounds represented in Table 1 were prepared according to the procedures disclosed by WO 2020/079184.

TABLE 1

| Compounds | Structure |
|---|---|
| MCK112 | 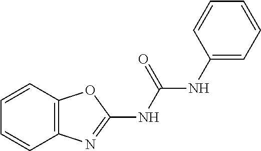 [SMILES: O=C(Nc1ccccc1)Nc1nc2ccccc2o1] |
| MCK113 | 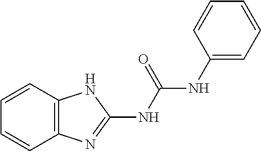 [SMILES: O=C(Nc1ccccc1)Nc1nc2ccccc2[nH]1] |
| MCK115 | 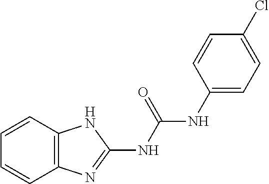 [SMILES: O=C(Nc1ccc(Cl)cc1)Nc1nc2ccccc2[nH]1] |
| MCK127 | 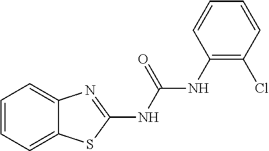 [SMILES: O=C(Nc1nc2ccccc2s1)Nc1ccccc1Cl] |
| MCK128 | 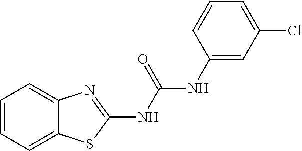 [SMILES: O=C(Nc1cccc(Cl)c1)Nc1nc2ccccc2s1] |
| MCK129 | 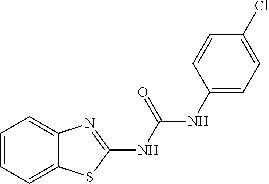 [SMILES: O=C(Nc1ccc(Cl)cc1)Nc1nc2ccccc2s1] |
| MCK130 | 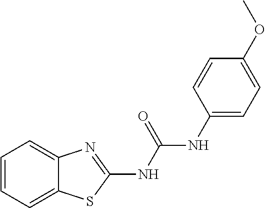 [SMILES: COc1ccc(NC(=O)Nc2nc3ccccc3s2)cc1] |
| MCK133 | 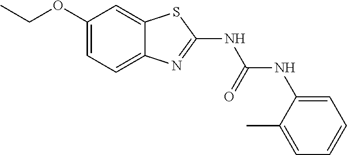 [SMILES: CCOc1ccc2nc(NC(=O)Nc3ccccc3C)sc2c1] |
| MCK136 | 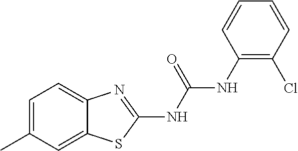 [SMILES: Cc1ccc2nc(NC(=O)Nc3ccccc3Cl)sc2c1] |
| MCK139 | 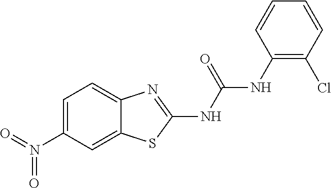 [SMILES: O=C(Nc1nc2ccc([N+](=O)[O-])cc2s1)Nc1ccccc1Cl] |
| MCK140 | 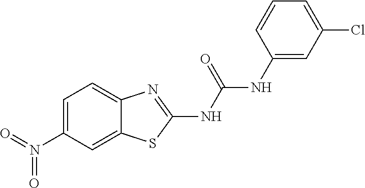 [SMILES: O=C(Nc1cccc(Cl)c1)Nc1nc2ccc([N+](=O)[O-])cc2s1] |
| MCK147 | 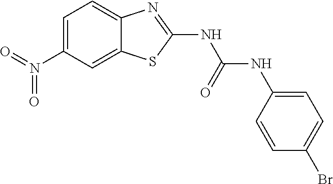 [SMILES: O=C(Nc1ccc(Br)cc1)Nc1nc2ccc([N+](=O)[O-])cc2s1] |
| MCK151 | 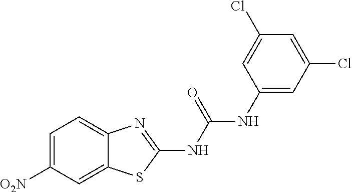 [SMILES: O=C(Nc1cc(Cl)cc(Cl)c1)Nc1nc2ccc([N+](=O)[O-])cc2s1] |
| MCK156 | 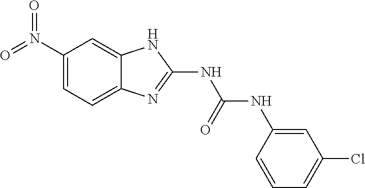 [SMILES: O=C(Nc1cccc(Cl)c1)Nc1nc2ccc([N+](=O)[O-])cc2[nH]1] |
| MCK165 | 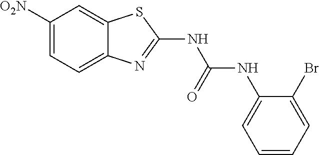 [SMILES: O=C(Nc1nc2ccc([N+](=O)[O-])cc2s1)Nc1ccccc1Br] |

### Chemical Synthesis and Characterization

Methanol, ethyl acetate, diethyl ether and dichloromethane were purchased from Carlo Erba, and use as received. Anhydrous DMF (99.8% stored under septum) was purchased from Sigma Aldrich, and use as received. All chemicals were purchased from Aldrich, Fisher or Alfa Aesar and used without further purification. Thin layer chromatography (TLC) was performed on precoated Merck 60 GF254 silica gel plates and revealed first by visualization under UV light (254 nm and 360 nm) ¹H and ¹³C NMR spectra were recorded on a Bruker Advance 200 MHz spectrometer or a Bruker Advance 400 MHz or a Bruker Advance 500 MHz. Mass spectra (ESI-MS) were recorded on a Bruker (Daltonics Esquire 3000+). HRMS spectra were recorded on a ThermoFisher Q Exactive (ESI-MS) at a resolution of 140 000 at m/z 200. The purity of compounds was further assayed by HPLC analysis on a JASCO PU-2089 apparatus with: Method 1: Supelco analytical column Ascentis Express C18, 100 mm×46 mm 5 μm. UV-detection: 214; 254; 280; 320 nm. Eluent A: water with 1% formic acid, Eluent B: CH₃CN with 1% formic acid. 0-1 min: 30% B; 1-6 min: 30-100% B; 6-8.5 min: 100% B; 8.5-9: 100-30% B; 9-13: 30% B. Method 2: Supelco analytical column Ascentis Express C18, 100 mm×46 mm 5 μm. UV-detection: 214; 254; 280; 320 nm. Eluent A: water with 1%% formic acid, Eluent B: CH₃CN with 1% formic acid. 0-1 min: 30% B; 1-6 min: 30-100% B; 6-8.5 min: 100% B; 8.5-9: 100-30% B; 9-16: 30% B. Method 3: Supelco analytical column Ascentis Express C18, 100 mm×46 mm 5 μm. UV-detection: 214; 254; 280; 320 nm. Eluent A: water with 1% formic acid, Eluent B: CH₃CN with 1% formic acid. 0-1 min: 30% B; 1-6 min: 30-100% B; 6-26 min: 100% B; 26-27: 100-30% B; 27-30: 30% B. Method 4: Supelco analytical column Ascentis Express C18, 100mm×46 mm 5 μm. UV-detection: 214; 254; 280; 320 nm. Eluent A: water with 1% formic acid, Eluent B: CH₃CN with 1% formic acid. 0-1 min: 30% B, 1-6 min: 30-100% B, 6-8.5 min: 100% B, 8.5-9 min: 100-30% B, 9-16: 30% B. Method 5: Waters Alliance 2695, Supelco Ascentis Express C18, 100mm×46 mm 5 μm. UV-detection: 214; 254; 280; 320 nm. Eluent A: water with 1% formic acid, Eluent B: CH₃CN with 1% formic acid. 0-10: 10% B; 10-18 min: 10-95% B; 18-20 min: 95% B; 20-24 min 95-10% B; 24-25 min: 10% B. Method 6: Ascentis Express C18, 100mm×46 mm 5 μM. UV-detection: 214; 254; 280; 360 nm. 0-1 min: 0% B; 1-10 min: 0-100% B; 10-15 min: 100% B

### Synthetic Procedures and Characterizations:

General procedure for the formation of ureas (A). To a solution of corresponding amine (1 equiv) in N,N-dimethylformamide (25 mL) was added the corresponding isocyanate (1 eq.) and the mixture was stirred overnight at room temperature. After completion of the reaction, the reaction mixture was poured into water (200 mL). The precipitate formed was collected and washed with methanol (2×25 mL) and diethyl ether (2×25 mL).

General procedure for the formation of ureas (B). To a solution of corresponding amine (1 equiv) in 1,4-dioxane (25 mL) was added the corresponding isocyanate (1 eq.) and the mixture was stirred overnight at room temperature. After completion of the reaction, the precipitate formed was collected and washed with methanol (2×25 mL) and diethyl ether (2×25 mL).

General procedure for the Suzuki reaction (C) To a solution of 1-(6-bromobenzo[d]thiazol-2-yl)-3-(3-chlorophenyl)urea (1 eq.) in a mixture of dioxane:water (4:1, 50 mL) was added the corresponding boronic acid (1.5 eq.), potassium carbonate (3 eq.), tetrakis triphenylphosphine palladium (0.1 eq.) and the mixture was refluxed overnight. The reaction mixture was concentrated under reduced pressure and the precipitate was extracted several times with hot isopropyl alcohol (10×50 mL). The alcoholic fractions were combined, concentrated under reduced pressure and purified by silica gel column chromatography (cyclohexane : EtOAc, 10:0 to 6:4).

General procedure for the formation of ureas (D). To a cooled (0° C.) solution of the corresponding aminobenzothiazole (1.1 eq.) in dry N,N-dimethylformamide (10-50 mL) was added dropwise the corresponding isocyanate (1 eq.). After completion of the addition, the ice bath was removed and the reaction mixture was stirred overnight at room temperature. After the reaction was complete, the precipitate formed was filtered, washed with water (2×25mL), diethyl ether (2×25 mL), and dried at air (56° C.).

1-(1H-benzo[d]imidazol-2-yl)-3-(3-chlorophenyl)urea (MCK109):

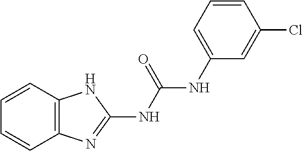 [SMILES: O=C(Nc1cccc(Cl)c1)Nc1nc2ccccc2[nH]1]

Synthesized following the general procedure (A) using benzo[d]thiazol-2-amine (500 mg, 3.33 mmol) and phenyl isocyanate (0.360mL, 3.33 mmol) to afford the title compound as a pink powder (117 mg, 12%); mp >260 ° C.; Rf (Cyclohexane/EtOAc, 75/25, v/v)=0.53; ¹H NMR (DMSO-d₆, 200 MHZ): δ (ppm): 7.35-7.10 (m, 4 H, H_{Ar}), 7.48-7.37 (m, 3 H, H_{Ar}), 7.92 (s, 1 H, H_{Ar}), 9.63 (s, 1 H, N-H), 11.49 (br.s, 2 H, 2N-H) ¹³C NMR (DMSO-d₆, 50 MHz) δ (ppm): 112.2 (2 C), 116.8 (2 C), 117.7 (2 C), 121.3, 121.5 130.3 (2 C), 133.1 (2 C), 141.3 150.1; ESI (m/z): [M+H]⁺ for C₁₄H₁₂ClN₄O⁺ 287.07, found 287.13; HPLC (λ₂₆₃Purity 83.0%,); t_{R}: 10.4 min (method 6).

1-(1H-benzo[d]imidazol-2-yl)-3-(2-chlorophenyl)urea (MCK110):

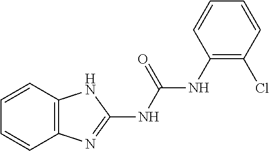 [SMILES: O=C(Nc1nc2ccccc2[nH]1)Nc1ccccc1Cl]

Synthesized following the general procedure (A) using benzo[d]thiazol-2-amine (500 mg, 3.33 mmol) and phenyl isocyanate (0.360 mL, 3.33 mmol) to afford the title compound as a grey powder (74.0 mg, 8%); mp >260° C.; Rf (Cyclohexane/EtOAc, 75/25, v/v)=0.50; ¹H NMR (DMSO-d₆, 200 MHz): δ (ppm) : 7.03-7.15 (m, 3 H, HAr), 7.29-7.43 (m, 3 H, HAr), 7.51 (dd, J=8 Hz, J=2 Hz, 1 H, H_{Ar}), 8.27 (d, J=8 Hz, 1 H, H_{Ar}), 10.21 (s, 1 H, N-H), 11.28 (s, 2 H, N-H). ESI (m/z): [M+H]⁺, calcd. for C₁₄H₁₂ClN₄O⁺287.07, found 287.06; HPLC (λ₂₆₃): Purity 68.2%; t_{R}: 9.5 min (method 6).

1-(benzo[d]thiazol-2-yl)-3-phenylurea (MCK126):

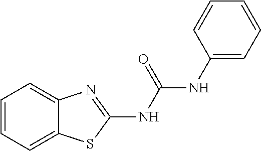 [SMILES: O=C(Nc1ccccc1)Nc1nc2ccccc2s1]

Synthesized following the general procedure (A) using benzo[d]thiazol-2-amine (500 mg, 3.33 mmol) and phenyl isocyanate (0.360 mL, 3.33 mmol) to afford the title compound as a white powder (830 mg, 93%). ¹H NMR (200 MHz, DMSO-d6): δ 10.88 (br. s, 1 H), 9.19 (s, 1 H), 7.91 (d, J=7.1 Hz, 1 H), 7.65 (d, J=7.8 Hz, 1 H), 7.55 (d, J=1.2 Hz, 2H), 7.45-7.29 (m, 3 H), 7.29-7.20 (m, 1 H), 7.06 (t, J=7.3 Hz, 1 H). ¹³C NMR (50 MHz, DMSO-d6) δ 159.81, 152.29, 147.93, 138.56, 131.17, 128.97 (2 C), 125.99, 122.99, 122.94, 121.54, 119.30, 118.89 (2 C). HRMS-ESI (m/z): [M+H]⁺calc. for C₁₄H₁₂N₃OS⁺, 270.06956; Found: 270.06961. HPLC (λ₂₈₀): Purity 95.4%; t_{R}: 9.708 min (method 6).

1-(1 H-benzo[d]imidazol-2-yl)-3-(4-methoxyphenyl)urea (MCK131):

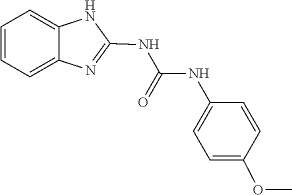 [SMILES: COc1ccc(NC(=O)Nc2nc3ccccc3[nH]2)cc1]

Synthesized following the general procedure (A) using 2-aminobenzimidazole (500 mg, 3.76 mmol) and 4-methoxyphenyl isocyanate (0.438 mL, 3.38 mmol) to afford the title compound as a white powder (922 mg, 87%). ¹H NMR (200 MHZ, DMSO-d6) δ 10.99 (br. s, 2 H), 9.46 (s, 1 H), 7.47 (d, J=8.7 Hz, 2 H), 7.37 (dd, J=5.5, 2.9 Hz, 2 H), 7.05 (dd, J=5.3, 2.9 Hz, 2 H), 6.90 (d, J=8.7 Hz, 2 H), 3.73 (s, 3 H). ¹³C NMR (50 MHz, DMSO-d6) δ 154.97, 153.62, 148.86, 135.58 (2 C), 132.24, 120.95 (2 C), 120.52 (2 C), 114.10 (2 C), 113.12 (2 C), 55.21. HRMS-ESI (m/z): [M+H]⁺ calc. for C₁₅H₁₅N₄O₂⁺, 283.11895; Found: 283.11902. HPLC (λ₂₈₀): Purity 97.9%; t_{R}: 6.433 min (method 6).

1-(6-ethoxybenzo[d]thiazol-2-yl)-3-(p-tolyl)urea (MCK132):

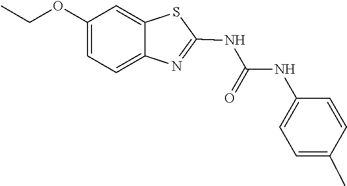 [SMILES: CCOc1ccc2nc(NC(=O)Nc3ccc(C)cc3)sc2c1]

Synthesized following the general procedure (A) using 2-amino-6-ethoxybenzothiazole (500 mg, 2.57 mmol) and p-tolyl isocyanate (0.324 mL, 2.57 mmol) to afford the title compound as a white powder (792 mg, 94%). ¹H NMR (200 MHZ, DMSO-d6): δ 10.63 (s, 1 H), 9.03 (s, 1 H), 7.54 (d, J=8.8 Hz, 1 H), 7.49 (d, J=2.5 Hz, 1 H), 7.39 (d, J=8.4 Hz, 2 H), 7.13 (d, J=8.3 Hz, 2 H), 6.96 (dd, J=8.8, 2.6 Hz, 1 H), 4.05 (q, J=6.9 Hz, 2 H), 2.26 (s, 3 H), 1.34 (t, J=7.0 Hz, 3 H). ¹³C NMR (50 MHz, DMSO-d6): δ 157.56, 154.94, 151.90, 142.41, 135.98, 132.52, 131.86, 129.34 (2 C), 120.07, 118.84 (2 C), 114.73, 105.53, 63.57, 20.39, 14.73. HRMS-ESI (m/z): [M+H]⁺ calc. for C₁₇H₁₈N₃O₂S⁺, 328.11142; Found: 328.11157. HPLC (λ₂₈₀): Purity 100.0%; t_{R}: 10.608 min (method 6).

1-(6-ethoxybenzo[d]thiazol-2-yl)-3-(m-tolyl)urea (MCK134):

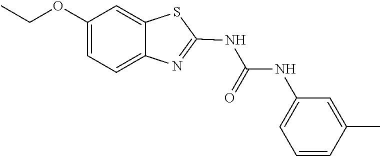 [SMILES: CCOc1ccc2nc(NC(=O)Nc3cccc(C)c3)sc2c1]

Synthesized following the general procedure (A) using 2-amino-6-ethoxybenzothiazole (500 mg, 2.57 mmol) and m-tolyl isocyanate (0.298 mL, 2.32 mmol) to afford the title compound as a white powder (731 mg, 87%). ¹H NMR (200 MHZ, DMSO-d6): δ 10.65 (br. s, 1 H), 9.05 (s, 1 H), 7.54 (d, J=8.8 Hz, 1 H), 7.50 (d, J=2.5 Hz, 1 H), 7.38-7.15 (m, 3 H), 6.96 (dd, J=8.8, 2.6 Hz, 1 H), 6.87 (d, J=6.9 Hz, 1 H), 4.05 (q, J=6.9 Hz, 2 H), 2.30 (s, 3 H), 1.34 (t, J=7.0 Hz, 3 H). ¹³C NMR (50 MHz, DMSO-d6): δ 157.57, 154.95, 151.86, 142.31, 138.46, 138.24, 132.49, 128.78, 123.65, 120.04, 119.25, 115.91, 114.79, 105.52, 63.59, 21.17, 14.73. HRMS-ESI (m/z): [M+H]⁺ calc. for C₁₇H₁₈N₃O₂S⁺, 328.11142; Found: 328.11151. HPLC (λ₂₈₀): Purity 97.5%; t_{R}: 10.617 min (method 6).

1-(6-ethoxybenzo[d]thiazol-2-yl)-3-(4-methoxyphenyl)urea (MCK135):

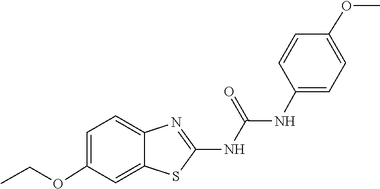 [SMILES: CCOc1ccc2nc(NC(=O)Nc3ccc(OC)cc3)sc2c1]

Synthesized following the general procedure (A) using 2-amino-6-ethoxybenzothiazole (500 mg, 2.57 mmol) and 4-methoxyphenylisocyanate (0.333 mL, 2.57 mmol) to afford the title compound as a white powder (775 mg, 88%). ¹H NMR (200 MHZ, DMSO-d6): δ 10.61 (br. s, 1 H), 8.95 (s, 1 H), 7.53 (d, J=8.8 Hz, 1 H), 7.49 (d, J=2.5 Hz, 1 H), 7.46-7.33 (m, 2 H), 7.01-6.85 (m, 3 H), 4.04 (q, J=6.9 Hz, 2 H), 3.73 (s, 3 H), 1.34 (t, J=6.9 Hz, 3 H). ¹³C NMR (50 MHz, DMSO-d6): δ 157.71, 155.25, 154.96, 152.03, 142.51, 132.58, 131.49, 120.72 (2 C), 120.12, 114.67, 114.10 (2 C), 105.53, 63.60, 55.17, 14.72. HRMS-ESI (m/z): [M+H]⁺ calc. for C₁₇H₁₈N₃O₃S⁺, 344.10634; Found: 344.10641. HPLC (λ₂₈₀): Purity 98.2%; t_{R}: 9.950 min (method 6).

1-(3-chlorophenyl)-3-(6-methylbenzo[d]thiazol-2-yl)urea (MCK137):

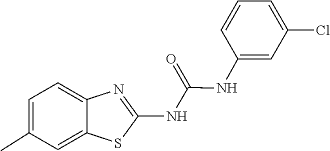 [SMILES: Cc1ccc2nc(NC(=O)Nc3cccc(Cl)c3)sc2c1]

Synthesized following the general procedure (A) using 6-methylbenzo[d]thiazol-2-amine (500 mg, 3.05 mmol) and 3-chlorophenyl isocyanate (0.364 mL, 3.05 mmol) to afford the title compound as a white powder (870 mg, 90%). ¹H NMR (400 MHZ, DMSO-d6): δ 11.06 (br. s, 1 H), 9.37 (s, 1 H), 7.76 (s, 1 H), 7.68 (s, 1 H), 7.51 (d, J=7.4 Hz, 1 H), 7.42-7.30 (m, 2 H), 7.20 (d, J=7.7 Hz, 1 H), 7.09 (d, J=6.9 Hz, 1 H), 2.38 (s, 3 H). ¹³C NMR (50 MHZ, DMSO-d6): δ 159.52, 152.91, 144.86, 140.33, 133.42, 132.36, 130.89, 130.39, 127.25, 122.44, 121.29, 118.41, 118.23, 117.23, 20.90. HRMS-ESI (m/z): [M+H]⁺ calc. for C₁₅H₁₃ClN₃OS⁺, 318.04624; Found: 318.04626. HPLC (λ₂₈₀): Purity 95.5%; t_{R}: 11.325 min (method 6).

1-(6-methylbenzo[d]thiazol-2-yl)-3-(m-tolyl)urea (MCK138):

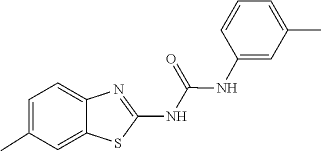

Synthesized following the general procedure (A) using 6-methylbenzo[d]thiazol-2-amine (500 mg, 3.05 mmol) and 3-tolylisocyanate (0.393 mL, 3.05 mmol) to afford the title compound as a white powder (788 mg, 87%). ¹H NMR (200 MHZ, DMSO-d6): δ 10.75 (br. s, 1 H, H10), 9.08 (s, 1 H, H12), 7.70 (s, 1 H, H3), 7.53 (d, J=8.1 Hz, 1 H, H6), 7.35 (s, 1 H, H19), 7.33-7.15 (m, 3 H, H1, H15, H19), 6.87 (d, J=7.2 Hz, 1 H, H17), 2.39 (s, 3 H, H20), 2.30 (s, 3 H, H21). ¹³C NMR (50 MHz, DMSO-d6): δ 158.91, 152.09, 146.09, 138.51, 138.24, 132.26, 131.34, 128.77, 127.17, 123.68, 121.19, 119.30, 119.04, 115.97, 21.18, 20.89. HRMS-ESI (m/z): [M+H]⁺ calc. for C₁₆H₁₆N₃OS⁺, 298.10086; Found: 298.10092. HPLC (λ₂₈₀): Purity 99.6%; t_{R}: 10.808 min (method 6).

1-(4-chlorophenyl)-3-(6-nitrobenzo[d]thiazol-2-yl)urea (MCK169):

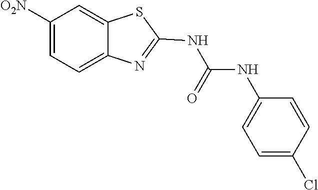 [SMILES: O=C(Nc1ccc(Cl)cc1)Nc1nc2ccc([N+](=O)[O-])cc2s1]

Synthesized following the general procedure (D) using 6-nitro-2-aminobenzothiazol (2.00 g, 13.30 mmol) in dry DMF and 4-chlorophenylisocyanate (1.85 g, 12.05 mmol) to afford the title compound as a yellow powder (2.88 g, 68%). ¹H NMR (200 MHz, DMSO-d6) δ 11.43 (s, 1 H), 9.37 (s, 1 H), 8.95 (s, 1 H), 8.23 (d, J=8.1 Hz, 1 H), 7.77 (d, J=8.9 Hz, 1 H), 7.55 (d, J=8.6 Hz, 2 H), 7.38 (d, J=8.5 Hz, 2 H). ¹³C NMR (101 MHz, DMSO-d6) δ: 164.8, 142.5, 138.5, 137.2, 133.7, 128.8 (2 C), 128.6, 126.9, 121.8, 120.6 (2 C), 119.8, 118.9. HRMS-ESI (m/z): [M+H]⁺ calcd for C₁₄H₁₀ClN₄O₃S, 349.0159; Found: 349.0157. HPLC (λ₂₅₄): Purity >99.9%; t_{R}: 12.27 min (method 5).

1-(6-nitrobenzo[d]thiazol-2-yl)-3-phenylurea (MCK155):

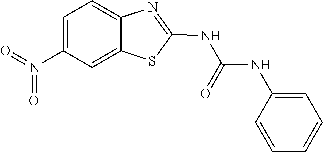 [SMILES: O=C(Nc1ccccc1)Nc1nc2ccc([N+](=O)[O-])cc2s1]

Synthesized following the general procedure (B) using 2-amino-6-nitrobenzothiazole (500 mg, 2.56 mmol) and phenyl isocyanate (0.28 mL, 2.56 mmol) to afford the title compound as a white powder (596 mg, 74%). ¹H NMR (200 MHZ, DMSO-d₆): δ 10.8 (br. s, 1 H), 9.46 (s, 1 H), 8.94 (d, J=2.3 Hz, 1 H), 8.21 (dd, J=8.9, 2.4 Hz, 1 H), 7.74 (d, J=8.9 Hz, 1 H), 7.54 (d, J=7.8 Hz, 2 H), 7.32 (t, J=7.8 Hz, 2 H), 7.06 (t, J=7.3 Hz, 1 H). ¹³C NMR (50 MHZ, DMSO-d₆): δ 164.97, 153.63, 151.98, 142.31, 138.34, 132.15, 128.84, 123.14, 121.62, 119.31, 119.06, 118.47. HRMS-ESI (m/z): [M+H]⁺ calc. for C₁₄H₁N₄O₃S⁺, 315.0746; Found: 315.0745. HPLC (λ₂₈₀): Purity 99.9%; t_{R}: 19.44 min (method 3).

1-(6-nitrobenzo[d]thiazol-2-ylidene)-3-(o-tolyl)urea (MCK173):

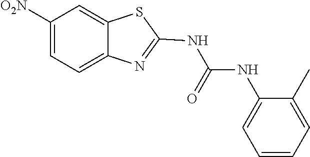 [SMILES: Cc1ccccc1NC(=O)Nc1nc2ccc([N+](=O)[O-])cc2s1]

Synthesized following the general procedure (D) using of 6-nitro-2-aminobenzothiazol (1.02 g, 6.79 mmol) and m-tolylisocyanate (892 mg, 6.70 mmol) to afford the title compound as a white powder (493.8 mg, 22%). ¹H NMR (400 MHZ, DMSO-d₆): δ 11.56 (s, 1 H), 9.00 (s, 1 H), 8.56 (s, 1 H), 8.25 (dd, J=8.9, 2.0 Hz, 1 H), 7.82 (d, J=7.2 Hz, 2 H), 7.23 (dd, J=15.2, 7.5 Hz, 2 H), 7.06 (t, J=7.3 Hz, 1 H), 2.28 (s, 3 H). ¹³C NMR (101 MHz, DMSO-d₆): δ 164.6, 153.9, 151.3, 142.3, 135.6, 132.1, 130.2, 128.4, 126.2, 124.0, 121.5, 121.4, 119.6, 118.5, 17.5. HRMS-ESI (m/z): [M+H]⁺ calc. for C₁₅H₁₃N₄O₃S⁺, 329.0703; Found: 329.0703, t_{R}: 10.78 min. HPLC (λ₂₅₄): Purity=98.59%; t_{R}: 11.85 min (method 5).

1-(6-nitrobenzo[d]thiazol-2-yl)-3-(p-tolyl)urea (MCK168):

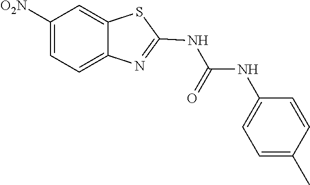 [SMILES: Cc1ccc(NC(=O)Nc2nc3ccc([N+](=O)[O-])cc3s2)cc1]

Synthesized following the general procedure (D) using of 6-nitro-2-aminobenzothiazol (2.03 g, 13.51 mmol) and p-tolylisocyanate (1.58 g, 11.87 mmol) to afford the title compound as a beige powder (2.21 g, 56%). ¹H NMR (200 MHZ, DMSO-d₆) δ 11.21 (s, 1 H), 9.10 (s, 1 H), 8.94 (s, 1 H), 8.22 (dd, J=8.9, 1.9 Hz, 1 H), 7.76 (d, J=8.8 Hz, 1 H), 7.39 (d, J=8.1 Hz, 2 H), 7.14 (d, J=8.1 Hz, 2 H), 2.26 (s, 3 H). ¹³C NMR (400 MHZ, DMSO-d₆) δ 165.07, 154.17, 151.76, 142.69, 135.70, 132.64, 132.38, 129.62 (2 C), 122.02, 119.69, 119.36 (2 C), 118.88, 20.64. HRMS-ESI (m/z): [M+H]⁺ calcd for C₁₅H₁₃N₄O₃S, 329.0705; Found: 329.0703. HPLC (λ₂₅₄): Purity>99.9%; t_{R}: 11.86 min (method 5).

1-(4-methoxyphenyl)-3-(6-nitrobenzo[d]thiazol-2-yl)urea (MCK152):

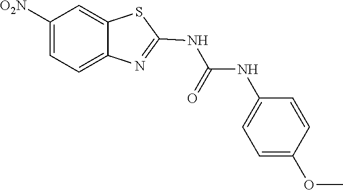 [SMILES: COc1ccc(NC(=O)Nc2nc3ccc([N+](=O)[O-])cc3s2)cc1]

Synthesized following the general procedure (D) using 6-nitro-2-aminobenzothiazol (2.06 g, 13.72 mmol) and 4-methoxyphenylisocyanate (1.50 mL, 12.09 mmol) to afford the title compound as a white powder (2.66g, 64%). ¹H NMR (200 MHZ, DMSO-d₆) δ 11.21 (br. s, 1 H), 8.96 (d, J=19.6 Hz, 2 H), 8.19 (d, J=6.5 Hz, 1 H), 7.74 (d, J=7.7 Hz, 1 H), 7.40 (d, J=6.3 Hz, 2 H), 6.92 (br. s, 2 H), 3.72 (s, 3 H). ¹³C NMR (50 MHz, DMSO-d₆) δ 164.81, 155.43, 153.59, 151.60, 142.33, 132.08, 130.86, 121.67, 120.90 (2 C), 119.36, 118.51, 114.05 (2 C), 55.15. HRMS-ESI (m/z): [M+H]⁺ calcd for C₁₅H₁₃N₄O₄S, 345.0654; Found: 345.0652 HPLC (λ₂₅₄): Purity>99.9%; t_{R}: 10.71 min (method 5).

1-(3-chlorophenyl)-3-(6-ethoxybenzo[d]thiazol-2-yl)urea (MCK166):

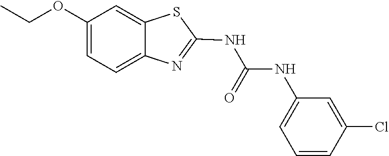 [SMILES: CCOc1ccc2nc(NC(=O)Nc3cccc(Cl)c3)sc2c1]

To a cooled (0° C.) solution of 2-amino-6ethoxybenzothiazole (1.00 g, 5.15 mmol, 1.1 eq.) in dry DMF (5.00 mL) was added dropwise 3-chloroisocyanate (623.1 mg, 4.68 mmol, 1 eq.). After completion of the addition, the ice bath was removed, and the reaction mixture was stirred at room temperature overnight. 1 more equivalent of 3-chloroisocyanate was added and after the reaction completion, Et₂O (10 mL) was added to the reaction mixture, followed by water (a white precipitate appeared). The white precipitate was washed with water (100 mL), then Et₂O (50 mL) to afford the title compound (1.68 g, quant.). ¹H NMR (200 MHZ, DMSO-d₆) δ 10.86 (br.s, 1 H), 9.34 (s, 1 H), 7.74 (s, 1 H), 7.52 (t, J=6.0 Hz, 2 H), 7.39-7.32 (m, 2 H), 7.15-7.03 (m, 1 H), 6.97 (dd, J=8.8, 2.6 Hz, 1 H), 4.05 (q, J=6.9 Hz, 2 H), 1.34 (t, J=6.9 Hz, 3 H). ¹³C NMR (101 MHz, DMSO-d₆) δ 158.81, 155.45, 152.94, 141.45, 140.69, 133.73, 132.57, 130.97, 122.92, 120.22, 118.60, 117.72, 115.31, 106.15, 64.06, 15.17. HRMS-ESI (m/z): [M+H]⁺ calcd for C₁₆H₁₅ClN₃O₂S, 348.0570; Found: 348.0568. HPLC (λ₂₅₄): Purity=98.0%; t_{R}: 12.76 min (method 5).

1-(3,5-dichlorophenyl)-3-(6-ethoxybenzo[d]thiazol-2-yl)urea (MCK167):

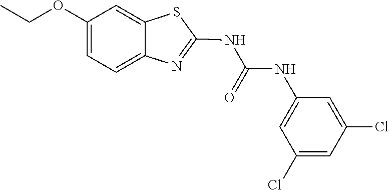 [SMILES: CCOc1ccc2nc(NC(=O)Nc3cc(Cl)cc(Cl)c3)sc2c1]

Synthesized following the general procedure (D) using 2-amino-6ethoxybenzothiazole (227.3 mg, 1.17 mmol) in dry DMF and 3,5-dichloroisocyanate (200.0 mg, 1.06 mmol) to afford the title compound as a white powder (1.68 g, quant.). ¹H NMR (200 MHZ, DMSO-d₆) δ 11.26 (br. s, 1 H), 9.52 (s, 1 H), 7.63 (d, J=1.7 Hz, 2 H), 7.50 (d, J=9.2 Hz, 2 H), 7.22 (s, 1 H), 6.97 (dd, J=8.7, 2.5 Hz, 1 H), 4.04 (q, J=6.8 Hz, 2 H), 1.34 (t, J=6.9 Hz, 3 H). ¹³C NMR (101 MHZ, DMSO-d₆) δ 159.8, 155.5, 154.2, 141.9, 134.6 (3 C), 132.1, 122.2, 119.4, 117.3 (2 C), 115.4, 106.3, 64.1, 15.2. HRMS-ESI (m/z): [M+H]⁺ calcd for C₁₆H₁₄Cl₂N₃O₂S, 382.0185; Found: 382.0178. HPLC (λ₂₅₄): Purity>99.9%; t_{R}: 14.66 min (method 5).

1-(3-chlorophenyl)-3-(6-nitrobenzo[d]oxazol-2-yl)urea (MCK160):

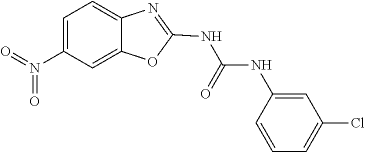 [SMILES: O=C(Nc1cccc(Cl)c1)Nc1nc2ccc([N+](=O)[O-])cc2o1]

Synthesized following the general procedure (B) using 6-nitrobenzo[d]oxazol-2-amine (500 mg, 2.80 mmol) and 3-chlorophenyl isocyanate (0.34 mL, 2.80 mmol) to afford the title compound as a white powder (492 mg, 53%). ¹H NMR (200 MHZ, DMSO-d₆): δ 12.19 (br. s, 1 H), 10.28 (s, 1 H), 8.46 (d, J=1.9 Hz, 1 H), 8.24 (dd, J=8.7, 2.2 Hz, 1 H), 7.81 (s, 1 H), 7.64 (d, J=8.7 Hz, 1 H), 7.54-7.41 (m, 1 H), 7.35 (t, J=8.0 Hz, 1 H), 7.11 (d, J=7.9 Hz, 1 H). ¹³C NMR (50 MHz, DMSO-d₆): δ 160.02, 145.52 (2 C), 142.72, 139.86 (2 C), 133.30, 130.27, 122.87, 121.18, 118.45, 117.42, 115.66, 106.08. HRMS-ESI (m/z): [M+H]⁺ calc. for C₁₄H₁₀ClN₄O₄⁺, 333.03851; Found: 333.03864. HPLC (λ₂₈₀): Purity 99.5%; t_{R}: 19.93 min (method 3).

1-(6-nitrobenzo[d]oxazol-2-yl)-3-(m-tolyl)urea (MCK153):

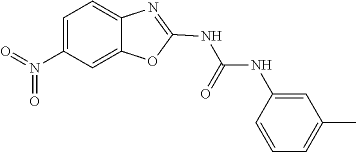 [SMILES: Cc1cccc(NC(=O)Nc2nc3ccc([N+](=O)[O-])cc3o2)c1]

Synthesized following the general procedure (B) using 6-nitrobenzo[d]oxazol-2-amine (500 mg, 2.80 mmol) and p-tolyl isocyanate (0.35 mL, 2.80 mmol) to afford the title compound as a white powder (419 mg, 48%). ¹H NMR (200 MHZ, DMSO-d₆) δ 11.95 (br. s, 1 H), 10.20 (s, 1 H), 8.52 (d, J=1.6 Hz, 1 H), 8.26 (dd, J=8.7, 2.2 Hz, 1 H), 7.71 (d, J=8.5 Hz, 1 H), 7.41 (d, J=6.6 Hz, 2 H), 7.24 (t, J=8.1 Hz, 1 H), 6.92 (d, J=7.3 Hz, 1 H), 2.31 (s, 3 H). ¹³C NMR (50 MHz, DMSO-d₆): δ 160.26, 150.12 (2 C), 145.91, 142.77, 138.22, 138.01, 128.75, 124.22, 121.27, 119.77, 116.46, 116.12, 106.32, 21.12. HRMS-ESI (m/z): [M+H]⁺ calc. for C₁₅H₁₃N₄O₄⁺, 313.09313; Found: 313.09320. HPLC (λ₂₅₄): Purity 95.3%; t_{R}: 21.09 min (method 3).

1-(3-chlorophenyl)-3-(5-nitrobenzo[d]oxazol-2-yl)urea (MCK158):

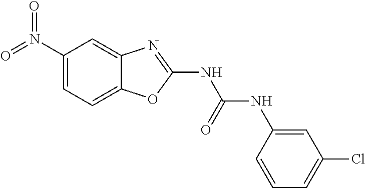 [SMILES: O=C(Nc1cccc(Cl)c1)Nc1nc2cc([N+](=O)[O-])ccc2o1]

Synthesized following the general procedure (B) using 5-nitrobenzo[d]oxazol-2-amine (500 mg, 2.80 mmol) and 3-chlorophenyl isocyanate (0.34 mL, 2.80 mmol) to afford the title compound as a white powder (633 mg, 68%). ¹H NMR (200 MHZ, DMSO-d₆): δ 11.91 (s, 1 H), 10.47 (s, 1 H), 8.36 (s, 1 H), 8.16 (dd, J=8.9, 2.4 Hz, 1 H), 7.82 (d, J=5.1 Hz, 2 H), 7.50 (d, J=8.8 Hz, 1 H), 7.36 (t, J=8.0 Hz, 1 H), 7.12 (d, J=7.6 Hz, 1 H). ¹³C NMR (50 MHz, DMSO-d₆): δ 8 158.71, 150.73 (2 C), 144.68, 140.91, 139.89, 133.29, 130.45, 122.81, 119.29, 118.27, 117.30, 112.73, 110.37 HRMS-ESI (m/z): [M+H]⁺ calc. for C₁₄H₁₀ClN₄O₄⁺, 333.03851; Found: 333.03879. HPLC (λ₂₈₀): Purity 99.6%; t_{R}: 19.97 min (method 3).

1-(5-nitrobenzo[d]oxazol-2-yl)-3-phenylurea (MCK154):

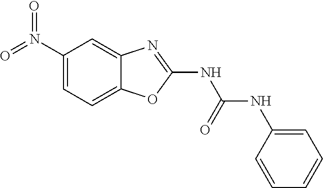 [SMILES: O=C(Nc1ccccc1)Nc1nc2cc([N+](=O)[O-])ccc2o1]

Synthesized following the general procedure (B) using 5-nitrobenzo[d]oxazol-2-amine (500 mg, 2.80 mmol) and phenyl isocyanate (0.30 mL, 2.80 mmol) to afford the title compound as a white powder (518 mg, 62%). ¹H NMR (200 MHZ, DMSO-d₆): δ 11.84 (s, 1 H), 10.46 (s, 1 H), 8.41 (s, 1 H), 8.17 (dd, J=8.9, 2.3 Hz, 1 H), 7.84 (d, J=8.9 Hz, 1 H), 7.58 (d, J=7.9 Hz, 2 H), 7.35 (t, J=7.8 Hz, 2 H), 7.08 (t, J=7.4 Hz, 1 H). ¹³C NMR (50 MHz, DMSO-d₆): δ 158.68, 151.03, 149.51, 144.75, 141.06, 138.29, 128.94, 123.30, 119.24, 118.95, 112.70, 110.38. HRMS-ESI (m/z): [M+H]⁺ calc. for C₁₄H₁₁N₄O₄⁺, 299.07748; Found: 299.07748. HPLC (λ₂₈₀): Purity 96.5%; t_{R}: 10.32 min (method 3).

1-(6-nitro-1 H-benzo[d]imidazol-2-yl)-3-phenylurea (MCK157):

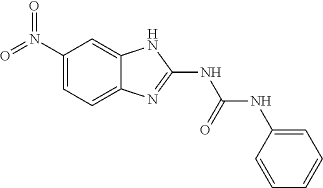 [SMILES: O=C(Nc1ccccc1)Nc1nc2ccc([N+](=O)[O-])cc2[nH]1]

Synthesized following the general procedure (B) using 6-nitro-1 H-benzo[d]imidazol-2-amine (500 mg, 2.80 mmol) and phenyl isocyanate (0.30 mL, 2.80 mmol) to afford the title compound as a white powder (433 mg, 52%). ¹H NMR (200 MHZ, DMSO-d₆): δ 11.48 (s, 2 H), 9.65 (s, 1 H), 8.28 (d, J=2.2 Hz, 1 H), 8.02 (dd, J=8.8, 2.3 Hz, 1 H), 7.67-7.47 (m, 3 H), 7.35 (t, J=7.9 Hz, 2 H), 7.06 (t, J=7.3 Hz, 1 H). ¹³C NMR (50 MHz, DMSO-d₆): δ 152.01, 151.63, 143.42, 141.49, 138.68, 135.51, 129.01 (2 C), 122.98, 118.76 (2 C), 117.26, 113.74, 109.23. HRMS-ESI (m/z): [M+H]⁺ calc. for C₁₄H₁₂N₅O₃⁺, 298.09347; Found: 298.09360. HPLC (λ₂₈₀): Purity 99.6%; t_{R}: 18.66 min (method 3).

1-(6-aminobenzo[d]thiazol-2-yl)-3-(3-chlorophenyl)urea (MCK148)

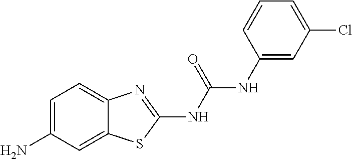 [SMILES: Nc1ccc2nc(NC(=O)Nc3cccc(Cl)c3)sc2c1]

To a cooled (0° C.) solution of 1-(3-chlorophenyl)-3-(6-nitrobenzo[d]thiazol-2-yl)urea (3.0 g, 8.6 mmol) in methanol (50 mL) was added palladium on charcoal (200 mg), then sodium borohydride (2.0 g, 52.8 mmol) portion wise. After the addition, the reaction mixture was allowed to warm to r.t. and stirred at this temperature until completion of the reaction (TLC monitoring, about 2 h). The mixture was filtered through celite© and the filtrate was concentrated under reduced pressure, then partitioned between water and EtOAc/MeOH, 9/1, v/v (3×80 mL). The combined organic layers were dried with Na₂SO₄ and evaporated. The solid obtained was triturated in EtOAc/Et₂O, 2/1, v/v, filtered and dried at air to afford the title compound as a pale yellow solid (950 mg, 35%). ¹H NMR (400 MHZ, DMSO-d₆): δ 10.77 (s, 1 H), 9.33 (s, 1 H), 7.75 (t, J=1.9 Hz, 1 H), 7.42-7.25 (m, 3 H), 7.07 (dt, J=7.5, 1.9 Hz, 1 H), 6.98 (d, J=2.1 Hz, 1 H), 6.68 (dd, J=8.6, 2.2 Hz, 1 H), 5.11 (s, 2 H). ¹³C NMR (101 MHZ, DMSO-d₆): δ 155.71, 152.72, 145.35, 140.43, 138.10, 133.29, 132.09, 130.51, 122.31, 119.37, 118.06, 117.17, 114.11, 104.60. HRMS-ESI (m/z): [M+H]⁺ calc. for C₁₄H₁₂ClN₄OS⁺, 319.04149; Found: 319.04169. HPLC (λ₂₅₄): Purity 98.3%; t_{R}: 7.26 min (method 4).

N-(2-(3-(3-chlorophenyl)ureido)benzo[d]thiazol-6-yl)acetamide (MCK149):

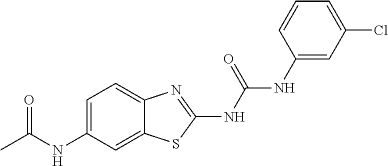 [SMILES: CC(=O)Nc1ccc2nc(NC(=O)Nc3cccc(Cl)c3)sc2c1]

To a cooled (0° C.) solution of 1-(6-aminobenzo[d]thiazol-2-yl)-3-(3-chlorophenyl)urea (96 mg, 0.30 mmol) in dichloromethane (2 mL) and DMF (2.5 mL), was added triethylamine (63 μL, 0.45 mmol), then acetic anhydride (34 μL, 0.36 mmol). After the addition, the reaction mixture was allowed to warm to r.t. and stirred at this temperature for 5 h. The reaction was quenched at 0° C. by addition of water (30 mL), then the mixture was extracted with CHCl₃/MeOH, 95/5, v/v. The combined organic layers were dried with Na₂SO₄ and evaporated. Purification by silica gel flash chromatography (dichloromethane/acetone, 9/1 to 5/5, v/v) afforded the desired compound as a pale yellow solid (79 mg, 74%). ¹H NMR (400 MHZ, DMSO-d₆) δ 11.00 (s, 1 H), 10.05 (s, 1 H), 9.36 (s, 1 H), 8.24 (d, J=1.7 Hz, 1 H), 7.74 (t, J=1.9 Hz, 1 H), 7.57 (d, J=8.7 Hz, 1 H), 7.45 (dd, J=8.7, 2.1 Hz, 1 H), 7.35 (dt, J=15.9, 8.3 Hz, 2 H), 7.09 (dt, J=7.7, 1.9 Hz, 1 H), 2.06 (s, 3 H). ¹³C NMR (101 MHZ, DMSO-d₆) δ 168.20, 158.95, 152.63, 143.18, 140.22, 135.00, 133.31, 131.25, 130.53, 122.52, 118.88, 118.30, 118.20, 117.29, 111.53, 23.98. HRMS-ESI (m/z): [M+H]⁺ calc. for C₁₆H₁₄ClN₄O₂S⁺, 361.05205; Found: 361.05228. HPLC (λ₂₅₄): Purity 98.3%; t_{R}: 7.26 min (method 4).

N-(2-(3-(3-chlorophenyl)ureido)benzo[d]thiazol-6-yl)methanesulfonamide (MCK150):

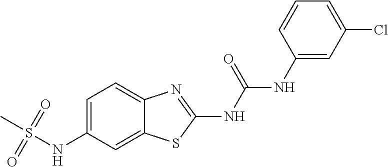 [SMILES: CS(=O)(=O)Nc1ccc2nc(NC(=O)Nc3cccc(Cl)c3)sc2c1]

To a cooled (0° C.) solution of 1-(6-aminobenzo[d]thiazol-2-yl)-3-(3-chlorophenyl)urea (96 mg, 0.30 mmol) in dichloromethane (2 mL) and DMF (2.5 mL) was added triethylamine (63 μL, 0.45 mmol), then mesyl chloride (28 μL, 0.36 mmol). The reaction mixture was allowed to warm to r.t. at the end of the addition and stirred at r.t. for 5 h. The reaction was quenched at 0° C. by addition of water (30 mL), then the mixture was extracted with CHCl₃/MeOH, 95/5, v/v. The combined organic layers were dried with Na₂SO₄ and evaporated. The residue was triturated with a little amount of dichloromethane/acetone, 1/1, v/v. The white precipitate was filtered to afford the title compound as a yellow solid (58 mg, 49%). ¹H NMR (400 MHZ, DMSO-d₆): δ 10.93 (s, 1 H), 9.72 (s, 1 H), 9.36 (s, 1 H), 7.74 (s, 2 H), 7.61 (d, J=8.5 Hz, 1 H), 7.41-7.32 (m, 2 H), 7.25 (dd, J=8.6, 2.2 Hz, 1 H), 7.10 (dd, J=8.9, 1.8 Hz, 1 H), 2.98 (s, 3 H). ¹³C NMR (50 MHz, DMSO-d₆): δ 159.47, 152.48, 144.47, 140.14, 133.59, 133.28, 131.75, 130.55, 122.59, 120.29, 119.53, 118.22, 117.35, 113.85, 39.78. HRMS-ESI (m/z): [M+H]⁺ calc. for C₁₅H₁₄ClN₄O₃S₂⁺, 397.01904; Found: 397.01904. HPLC (λ₂₅₄): Purity 98.5%; t_{R}: 7.46 min (method 4).

1-(6-bromobenzo[d]thiazol-2-yl)-3-(3-chlorophenyl)urea (MCK159):

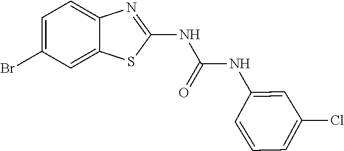 [SMILES: O=C(Nc1cccc(Cl)c1)Nc1nc2ccc(Br)cc2s1]

Synthesized following the general procedure (B) using 6-bromobenzo[d]thiazol-2-amine (500 mg, 2.18 mmol) and 3-chlorophenyl isocyanate (0.27 mL, 2.18 mmol) to afford the title compound as a white powder (685 mg, 82%). ¹H NMR (200 MHZ, DMSO-d₆): δ 11.28 (br. s, 1 H), 9.45 (s, 1 H), 8.18 (s, 1 H), 7.74 (s, 1 H), 7.66-7.45 (m, 2 H), 7.46-7.26 (m, 2 H), 7.10 (d, J=6.6 Hz, 1 H). ¹³C NMR (50 MHz, DMSO-d₆): δ 160.49, 152.38, 146.83, 140.01, 133.39, 133.31, 130.47, 128.97, 124.04, 122.69, 120.75, 118.33, 117.35, 114.91. HRMS-ESI (m/z): [M+H]⁺ calc. for C₁₄H₁₀BrClN₃OS⁺, 381.94110; Found: 381.94110. HPLC (λ₂₈₀): Purity 99.9%; t_{R}: 19.44 min (method 3).

1-(3-chlorophenyl)-3-(6-(furan-2-yl)benzo[d]thiazol-2-yl)urea (MCK162):

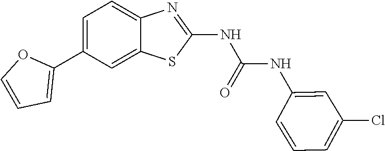 [SMILES: O=C(Nc1cccc(Cl)c1)Nc1nc2ccc(-c3ccco3)cc2s1]

Synthesized following the general procedure (C) using 1-(6-bromobenzo[d]thiazol-2-yl)-3-(3-chlorophenyl)urea (1.00 g, 2.61 mmol), 2-furanylboronic acid (439 mg, 3.92 mmol), potassium carbonate (1.08 g, 7.83 mmol) and tetrakis triphenylphosphine palladium (300 mg, 0.261 mmol) to afford the title compound as a white powder (473 mg, 49%). ¹H NMR (200 MHZ, DMSO-d₆): δ 11.04 (s, 1 H), 9.40 (s, 1 H), 8.26 (s, 1 H), 7.70 (dd, J=17.4, 6.9 Hz, 4 H), 7.52-7.26 (m, 2 H), 7.11 (d, J=6.7 Hz, 1 H), 6.95 (d, J=3.1 Hz, 1 H), 6.61 (s, 1 H). ¹³C NMR (50 MHz, DMSO-d₆): δ 160.34, 153.00, 152.81, 146.61, 142.66, 140.14, 133.28, 131.71, 130.52, 125.62, 122.58, 122.03, 119.16, 118.23, 117.34, 116.50, 112.15, 105.35. HRMS-ESI (m/z): [M+H]⁺ calc. for C₁₈H₁₃ClN₃O₂S⁺, 370.04115; Found: 370.04099. HPLC (λ₂₈₀): Purity 98.8%; t_{R}: 21.00 min (method 3).

1-(3-chlorophenyl)-3-(6-(thiophen-2-yl)benzo[d]thiazol-2-yl)urea (MCK161):

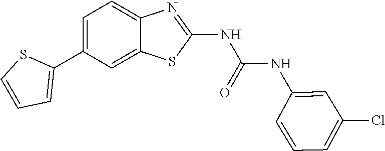 [SMILES: O=C(Nc1cccc(Cl)c1)Nc1nc2ccc(-c3cccs3)cc2s1]

Synthesized following the general procedure (C) using 1-(6-bromobenzo[d]thiazol-2-yl)-3-(3-chlorophenyl)urea (1.00 g, 2.61 mmol), 2-thienylboronic acid (502 mg, 3.92 mmol), potassium carbonate (1.08 g, 7.83 mmol) and tetrakis triphenylphosphine palladium (300 mg, 0.261 mmol) to afford the title compound as a white powder (383 mg, 38%). ¹H NMR (200 MHZ, DMSO-d₆): δ 11.02 (s, 1 H), 9.40 (s, 1 H), 8.25 (s, 1 H), 7.72 (d, J=17.1 Hz, 3 H), 7.53 (d, J=4.3 Hz, 2 H), 7.43-7.28 (m, 2 H), 7.13 (dd, J=9.1, 5.3 Hz, 2 H). ¹³C NMR (101 MHz, DMSO-d₆): δ 160.22, 152.59, 145.96, 143.28, 140.33, 133.25, 130.82, 130.55, 128.96, 128.52, 125.34, 124.00, 123.44, 122.61, 122.54, 118.38, 118.22, 117.36. HRMS-ESI (m/z): [M+H]* calc. for C₁₈H₁₃ClN₃OS₂⁺, 386.01831; Found: 386.01810. HPLC (λ280): Purity 95.1%; t_{R}: 12.48 min (method 3).

1-(3-chlorophenyl)-3-(6-(thiophen-3-yl)benzo[d]thiazol-2-yl)urea (MCK163):

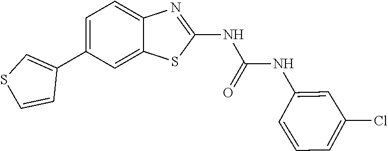 [SMILES: O=C(Nc1cccc(Cl)c1)Nc1nc2ccc(-c3ccsc3)cc2s1]

Synthesized following the general procedure (C) using 1-(6-bromobenzo[d]thiazol-2-yl)-3-(3-chlorophenyl)urea (1 g, 2.61 mmol), 3-thienylboronic acid (501 mg, 3.92 mmol), potassium carbonate (1.08 g, 7.83 mmol) and tetrakis triphenylphosphine palladium (300 mg, 0.261 mmol) to afford the title compound as a white powder (423 mg, 42%). ¹H NMR (200 MHZ, DMSO-d₆): δ 11.12 (br. s, 1 H), 9.41 (s, 1 H), 8.29 (s, 1 H), 7.89 (s, 1 H), 7.83-7.73 (m, 2 H), 7.71-7.55 (m, 3 H), 7.37 (q, J=8.3 Hz, 2 H), 7.11 (d, J=6.9 Hz, 1 H). ¹³C NMR (50 MHZ, DMSO-d₆): δ 160.3, 152.8, 146.1, 141.2, 140.2, 133.3, 131.6, 130.5, 130.4, 127.0, 126.2, 124.5, 122.5, 120.4, 118.9 (2 C), 118.2, 117.3. HRMS-ESI (m/z): [M+H]⁺ calc. for C₁₈H₁₃ClN₃OS₂⁺, 386.01831; Found: 386.01877. HPLC (λ₂₈₀): Purity 99.8%; t_{R}: 21.15 min (method 3).

1-(3-chlorophenyl)-3-(6-(naphthalen-1-yl)benzo[d]thiazol-2-yl)urea (MCK164):

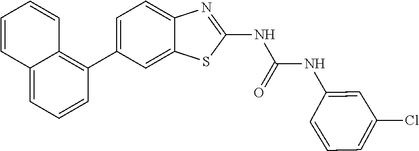 [SMILES: O=C(Nc1cccc(Cl)c1)Nc1nc2ccc(-c3cccc4ccccc34)cc2s1]

Synthesized following the general procedure (C) using 1-(6-bromobenzo[d]thiazol-2-yl)-3-(3-chlorophenyl)urea (1.00 g, 2.61 mmol), naphthalene-1-boronic acid (674 mg, 3.92 mmol), potassium carbonate (1.08 g, 7.83 mmol) and tetrakis triphenylphosphine palladium (300 mg, 0.261 mmol) to afford the title compound as a white powder (583 mg, 52%). ¹H NMR (200 MHz, DMSO-d₆): δ 11.09 (br. s, 1 H), 9.45 (s, 1 H), 8.13-7.92 (m, 3 H), 7.83 (dd, J=10.5, 8.9

Hz, 3 H), 7.65-7.31 (m, 7 H), 7.11 (d, J=7.2 Hz, 1 H). ¹³C NMR (50 MHz, DMSO-d₆): δ 160.7, 152.8, 146.2, 140.2, 139.3, 134.9, 133.5, 133.3, 131.0 (2 C), 130.5, 128.3, 128.1, 127.6, 127.2, 126.4, 125.9, 125.5, 125.4, 122.7, 122.5, 118.4, 118.3, 117.3. HRMS-ESI (m/z): [M+H]⁺ calc. for C₂₄H₁₇ClN₃OS⁺, 430.07754; Found: 430.07764. HPLC (λ₂₈₀): Purity 97.3%; t_{R}: 22.33 min (method 3).

1-(3-chlorophenyl)-3-(6-(pyridin-2-yl)benzo[d]thiazol-2-yl)urea (MCK172):

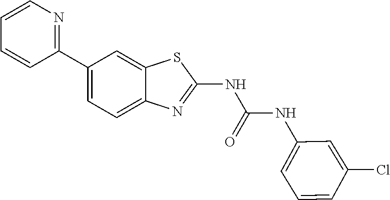 [SMILES: O=C(Nc1cccc(Cl)c1)Nc1nc2ccc(-c3ccccn3)cc2s1]

To a solution of (Z)-N′-(6-bromobenzo[d]thiazol-2-yl)-N-(3-chlorophenyl)carbamimidic acid (500.00 mg, 1.30 mmol, leq) and pyridin-2-ylboronic acid (239.69 mg, 1.95 mmol, 1.5 eq) in a mixture of degassed dioxane/water (24 mL/6 mL), was added K₂CO₃ (719 mg, 5.2 mmol, 4 eq) followed by Pd(PPh₃)₄ (152 mg, 0.13 mmol, 0.1 eq.). The reaction mixture was stirred overnight at reflux temperature, then cooled down to r.t. and partitioned between water and EtOAc. The aqueous phase was extracted with EtOAc and the combined organic layers were dried with Na₂SO₄ and concentrated under reduced pressure to afford a brown-orange residue. Purification by silica gel flash chromatography (CHCl₃/MeOH, 100/0 to 95/5, v/v) afforded the title compound as beige solid (494 mg, 22%). ¹H NMR (400 MHZ, DMSO-d₆) δ 11.15 (br. s, 1 H), 9.43 (s, 1 H), 8.96 (s, 1 H), 8.56 (d, J=4.0 Hz, 1 H), 8.33 (s, 1 H), 8.13 (d, J=8.1 Hz, 1 H), 7.76 (s, 3 H), 7.49 (dd, J=7.8, 4.8 Hz, 1 H), 7.38 (dt, J=15.9, 7.9 Hz, 2 H), 7.11 (d, J=7.2 Hz, 1 H). ¹³C NMR (101 MHz, DMSO-d₆) δ 160.8, 149.2, 148.2, 147.8, 147.7, 140.2, 135.4, 134.5, 134.1, 133.3, 132.0, 130.6, 125.2, 123.9, 122.6, 120.1, 119.4, 118.3, 117.4. HRMS-ESI (m/z): [M+H]+calc. for C₁₉H₁₄ClN₄OS⁺, 381.0571; Found: 381.0574. HPLC (λ₂₅₄): Purity 96.3%; t_{R}: 8.02 min (method 5).

### Example B: Biology

### Material and Methods:

Cell culture: Uveal melanoma cells were derived from the primary tumor (eye tumor, MP38 and MP46) and one is from liver metastasis (MM66) were a kind gift from Dr. Roman Roman (Curie Institute). UM cells were cultured in RPMI medium supplemented with 20% FBS.

Cell viability (XTT): Cells (5×10³ cells/100 μl) were incubated in a 96-well plate with different effectors for the times indicated in the figure legends. 50 μl of sodium 3′-[1-phenylaminocarbonyl)-3,4-tetrazolium]-bis(4-methoxy-6-nitro) benzene sulfonic acid hydrate (XTT) reagent was added to each well. The assay is based on the cleavage of the yellow tetrazolium salt XTT to form an orange formazan dye by metabolically active cells. Absorbance of the formazan product, reflecting cell viability, was measured at 490 nm. Each assay was performed in quadruplicate.

Immunoblotting: Cells were lysed in SDS 7.5%; glycerol 30%; Tris 0.3 M pH 6.8 lysis buffer. 30 μg of proteins were separated on 10% SDS—polyacrylamide gels and transferred on PVDF membranes. The following primary antibodies were used: SLUG (Cell signaling, ref:9585) and HSP90 (Cell signaling, ref:4877).

Analyses by RT-qPCR: RNA from cells were purified with the RNeasy Mini Kit (Quiagen). The “QuantiTect Reverse Transcription Kit” (Qiagen) was used for cDNA obtention. The PCR program was executed on “Professional Basic Thermocycler” (Biometra). SYBR master mix plus (Eurogentec) was used for qPCR. The mRNA levels were normalized to 36B4 mRNA.

Migration assays: 50 000 cells were cultured in RPMI 0% FCS and seeded in Boyden chambers. After 24 h, Boyden chambers were washed with PBS. Migrative cells were fixed with paraformaldehyde 3% and colored with crystal violet.

ROS assay: Deep Red Reagent was used to assess the level of intracellular ROS. The CellROX/Deep Red reagent (which is initially non-fluorescent) freely enters the cells, where it is cleaved by endogenous esterases. After oxidization by ROS, the reagent becomes highly fluorescent with an absorption/emission maximal of 644/665 nm. Cells were treated 24h and incubated at 37° C. with CellROX Deep Red reagent (0.01 umol/L) for 2 h. The cells were then washed with PBS and analyzed by cytometry.

Tumor xenograft experiments: These studies were carried out in strict accordance with the recommendations in the Guide for the Care and Use of Laboratory Animals. Our experiments were approved by the “Comité National Institutionnel d'Ethique pour l'Animal de Laboratoire”. Five million MP41 cells were injected subcutaneously into the flank of 5-week-old Nod-SDIC female mice (Janvier). When the tumor reached 100 mm3, mice were treated. The tumor volume was determined with a caliper (v=L*l2*0.5).

### Results:

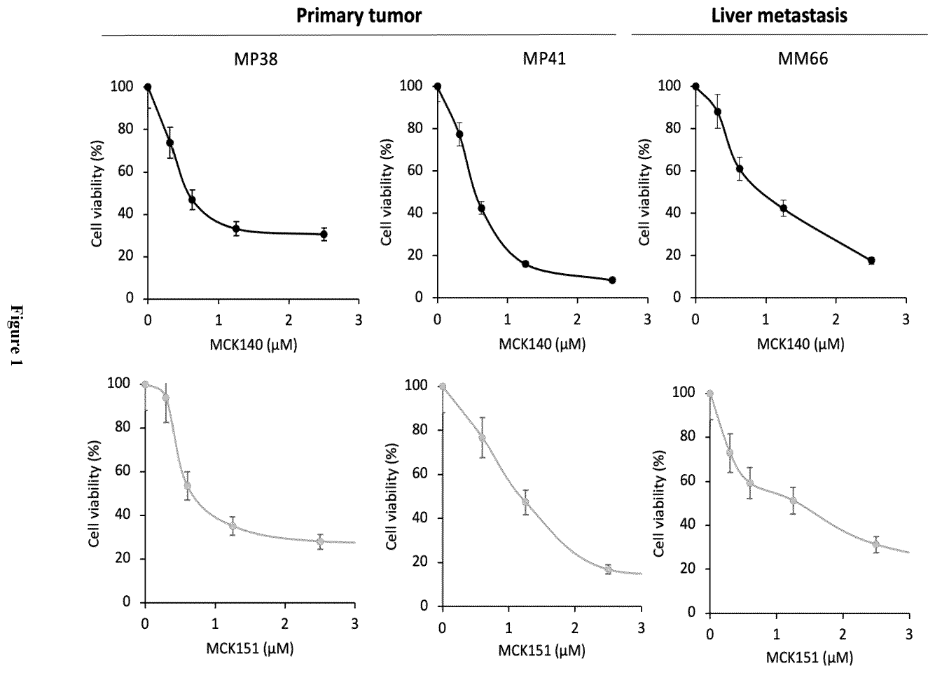

Results are illustrated by FIGS. 1-5, Tables 2 and 3 below.

In vitro efficacy of MCK compounds in comparison with other CXCR1 or/and CXCR₂ inhibitors on uveal melanoma cells.

TABLE 2

| IC50 (μM) | UM | | |
|---|---|---|---|
| MCK | MP38 | MP41 | MM66 |
| 140 | 1 | 1 | 1.25 |
| 151 | 1 | 1.25 | 1.5 |

MCK140 and MCK151 decreased the viability of cells from the primary tumor (MP38 and MP41) and from liver metastases (MM66).

TABLE 3
MCK compounds of the invention decrease the viability
of cells from the primary tumor (MP41) and from
liver metastases (MM66). IC50 (μM) at 48 h.

| MCK compounds | MP41 | MM66 |
|---|---|---|
| 113 | 32 | 40 |
| 133 | 15 | 20 |
| 135 | 50 | 50 |
| 139 | 20 | 15 |
| 140 | 1 | 1.25 |
| 148 | 50 | 50 |
| 149 | 40 | 25 |
| 151 | 1.25 | 1.5 |
| 153 | 30 | 12 |
| 157 | 8 | 10 |
| 159 | 20 | 10 |
| 164 | 10 | 5 |
| 172 | 30 | 25 |

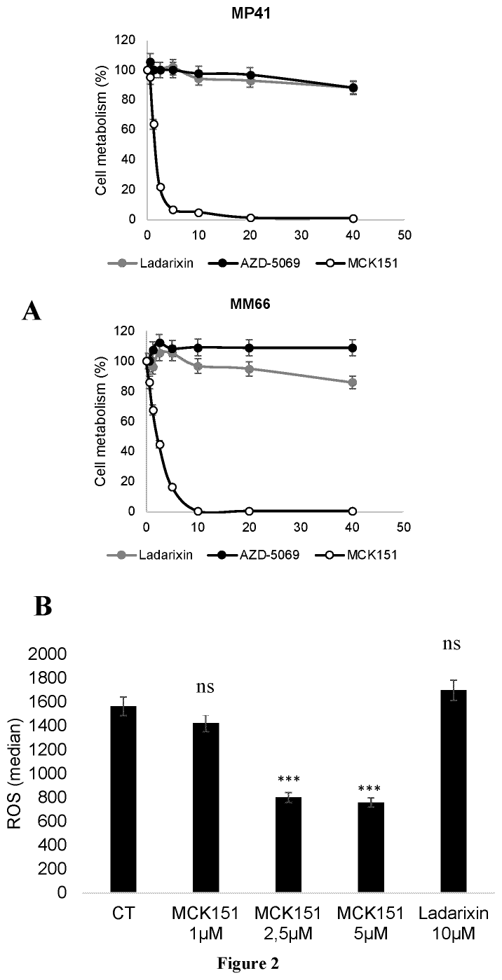

The efficacy of MCK151 on uveal melanoma cells was compared to other CXCR1 and/or CXCR₂ inhibitors (Ladarixin and AZD-5069). The results show that MCK151 strongly inhibits the metabolism of UM cells whereas AZD-5069 and Ladarixin have no effect on UM cells (FIG. 2A).

Inhibition of ROS production by MCK151 and other CXCR1 or/and CXCR₂ inhibitors UM cells.

Cancer cells, as a result of hypermetabolism, have higher levels of reactive oxygen species (ROS) as compared to normal cells. ROS are also implicated in tumorigenesis (tumor initiation, tumor progression, and metastasis) and favor the aggressiveness of cancer cells and, more particularly, for UM. The inventors have shown that MCK151 inhibits ROS production by UM cells whereas ladarixin has no effect (FIG. 2B).

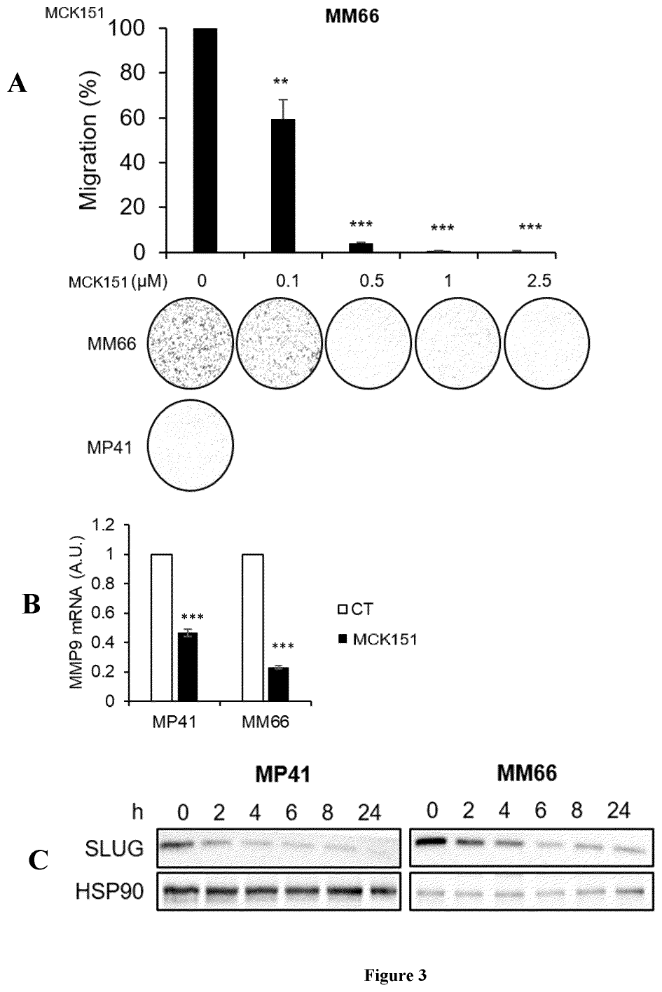

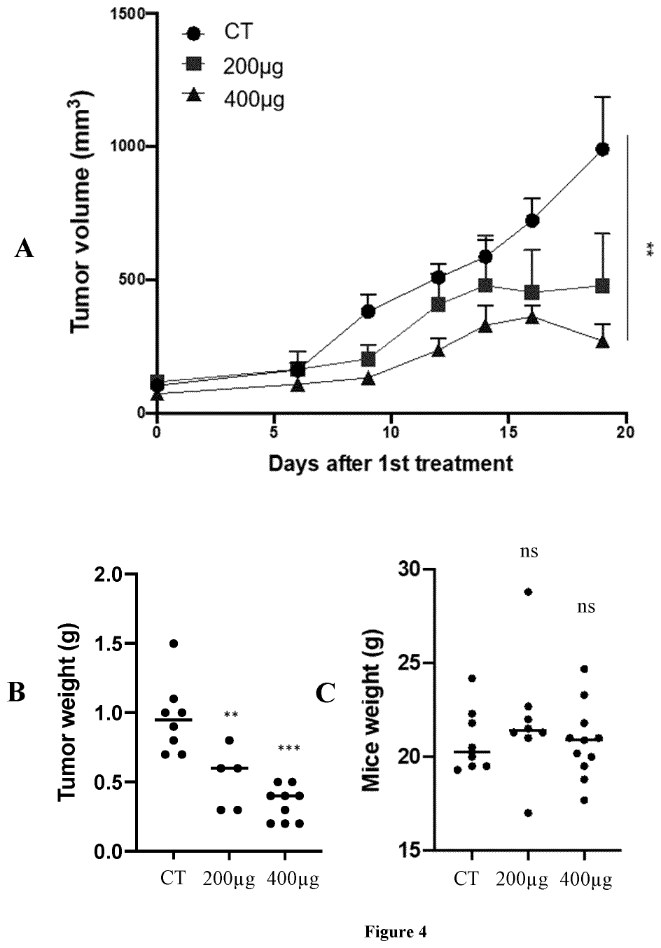

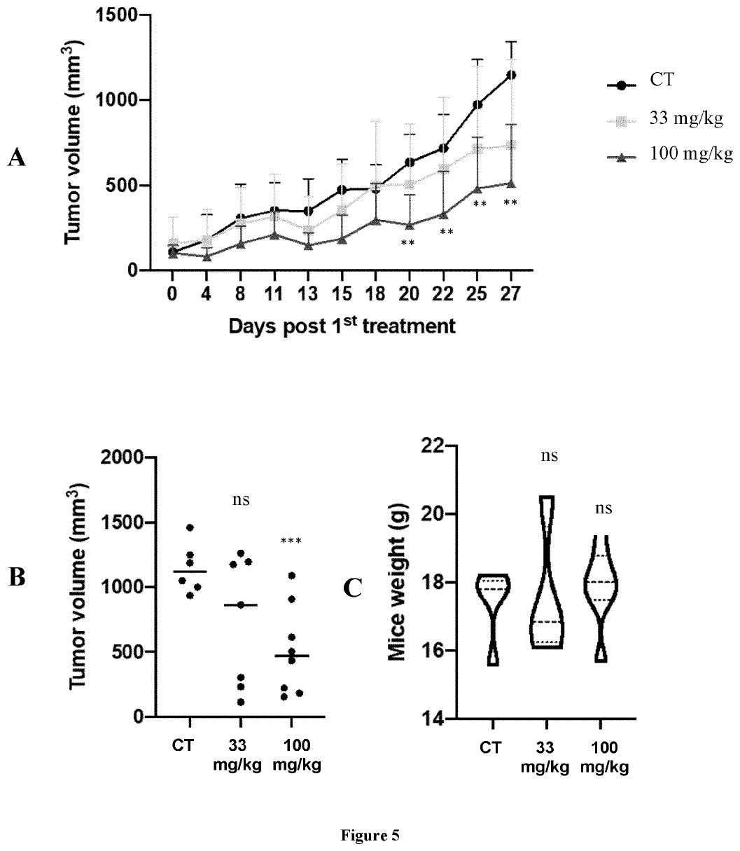

Evaluation of the migration ability of uveal melanoma cells treated with MCK151.

Whereas MP41 cells isolated from a primary tumor do not migrate, MM66 cells, isolated from a liver metastasis, have this property. MCK151 inhibits this migration ability with an optimal effect at 0.5 μM (FIG. 3A). Moreover, MCK151 decreases epithelial-mesenchymal transition

(EMT) markers. Indeed, MCK151 decreases MMP9 mRNA levels (FIG. 3B) and the expression of SLUG (FIG. 3C). Altogether, these results show that MCK compounds, in particular MCK151, are efficient for the treatment of primary and metastatic UM.

In vivo efficacy of MCK151 on mice treated by intraperitoneal injection.

Inventors have evaluated the efficacy of MCK151 in vivo. The growth of experimental tumors generated with MP41 cells in immunodeficient NOD-SCID mice was inhibited by MCK151. in a dose-dependent manner (inhibition of 73% with the 400 μg dose)—FIG. 4A. At the end of the experiment, the tumors from mice treated with MCK151 were smaller (FIG. 4B). Importantly, MCK151 did not modify the weight of mice whatever the concentration (FIG. 4C).

In vivo efficacy of MCK151 on mice treated by gavage.

An oral formulation (10% ethanol in ultrapure water) was developed and tested in the same in vivo model (FIG. 5). MCK151 orally administered at 33 mg/kg per day, had a trend to decrease tumor growth. However, a 100 mg/kg dose reduced tumor growth (50% reduction, FIG. 5A). Tumor weight was also reduced by 50% (FIG. 5B). At 33 and 100 mg/kg/day, MCK151 did not modify mice weight (FIG. 5C). These results show that the MCK compounds of the invention, in particular MCK151 can be used by oral route.

1-15. (canceled)

16. A method of treating uveal melanoma comprising administering a compound of formula (I), a pharmaceutically acceptable salt thereof, a tautomer thereof, or a composition comprising said compound, pharmaceutically acceptable salt, or tautomer to a subject having uveal melanoma: 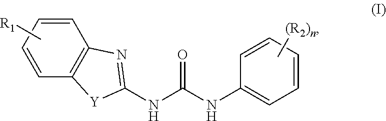
in which:
Y is 13 NH—, —S—, or —O—;
R₁ is a radical selected from the group consisting of:
a hydrogen atom,
a (C₁-C₆)alkyloxy group,
a (C₁-C₆)alkyl group,
a nitro group,
a —NR₃R₄ group with R₃ and R₄ are independently a radical selected from the group consisting of:
a hydrogen atom,
a —COR₅ with R₅ is a (C₁-C₆)alkyl group, and
a —SO₂R₆ with R₆ is a (C₁-C₆)alkyl group,
a halogen atom, and
a 3-14 membered ring selected from the group consisting of an aryl, a heteroaryl, a cycloalkyl, and a heterocycloalkyl, said 3-14 membered ring is optionally substituted by a radical selected from the group consisting of a (C₁-C₆)alkyl group, a (C₁-C₆)alkyloxy group, a halogen atom, a nitro group, and a carboxyl group;
R₂ is a radical selected from the group consisting of:
a hydrogen atom,
a halogen atom,
a (C₁-C₆)alkyloxy group, and
a (C₁-C₆)alkyl group;
n is an integer number from 0 to 5.

17. The method according to claim 16, wherein Y is —S— or —O—.

18. The method according to claim 16, wherein Ri is a radical selected from the group consisting of:
a hydrogen atom,
an ethoxy group,
a methyl group,
a nitro group,
a —NR₃R₄ group with R₃ and R₄ are independently a radical selected from the group consisting of:
a hydrogen atom,
a —COR₅ with R₅ is a methyl group, and
a —SO₂R₆ with R₆ is a methyl group,
a halogen atom, and
a 3-14 membered ring selected from the group consisting of a thiophenyl, a furanyl, a naphthalenyl, and a pyridinyl, said 3-14 membered ring is optionally substituted by a radical selected from the group consisting of a (C₁-C₆)alkyl group, a (C₁-C₆)alkyloxy group, a halogen atom, a nitro group, and a carboxyl group.

19. The method according to claim 16, wherein R₁ is a radical selected from the group consisting of an ethoxy group, a nitro group, and a 3-14 membered ring selected from the group consisting of a thiophenyl, a furanyl, a naphthalenyl, and a pyridinyl, said 3-14 membered ring is optionally substituted by a radical selected from the group consisting of a (C₁-C₆)alkyl group, a (C₁-C₆)alkyloxy group, a halogen atom, a nitro group, and a carboxyl group.

20. The method according to claim 16, wherein n is an integer number from 1 to 3.

21. The method according to claim 16, wherein n is 1 or 2, and R₂ is in meta position.

22. The method according to claim 16, wherein R₂ is a radical selected from the group consisting of:
a hydrogen atom,
a halogen atom,
a methoxy group, and
a methyl group.

23. The method according to claim 16, wherein said compound is of formula (IA):

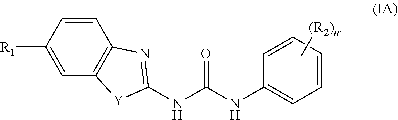

24. The method according to claim 16, wherein said compound is selected from the group consisting of:
1-(1H-benzo[d]imidazol-2-yl)-3-(3-chlorophenyl)urea (MCK109);
1-(1H-benzo[d]imidazol-2-yl)-3-(2-chlorophenyl)urea (MCK110);
1-(benzo[d]oxazol-2-yl)-3-phenylurea (MCK112);
1-(1H-benzo[d]imidazol-2-yl)-3-phenylurea (MCK113);
1-(1H-benzo[d]imidazol-2-yl)-3-(4-chlorophenyl)urea (MCK115);
1-(benzo[d]thiazol-2-yl)-3-phenylurea (MCK126);
1-(benzo[d]thiazol-2-yl)-3-(2-chlorophenyl)urea (MCK127);
1-(benzo[d]thiazol-2-yl)-3-(3-chlorophenyl)urea (MCK128);
1-(benzo[d]thiazol-2-yl)-3-(4-chlorophenyl)urea (MCK129);
1-(benzo[d]thiazol-2-yl)-3-(4-methoxyphenyl)urea (MCK130);
1-(1H-benzo[d]imidazol-2-yl)-3-(4-methoxyphenyl)urea (MCK131);
1-(6-ethoxybenzo[d]thiazol-2-yl)-3-(p-tolyl)urea (MCK132);
1-(6-ethoxybenzo[d]thiazol-2-yl)-3-(o-tolyl)urea (MCK133);
1-(6-ethoxybenzo[d]thiazol-2-yl)-3-(m-tolyl)urea (MCK134);
1-(6-ethoxybenzo[d]thiazol-2-yl)-3-(4-methoxyphenyl)urea (MCK135);
1-(2-chlorophenyl)-3-(6-methylbenzo[d]thiazol-2-yl)urea (MCK136);
1-(3-chlorophenyl)-3-(6-methylbenzo[d]thiazol-2-yl)urea (MCK137);
1-(6-methylbenzo[d]thiazol-2-yl)-3-(m-tolyl)urea (MCK138);
1-(2-chlorophenyl)-3-(6-nitrobenzo[d]thiazol-2-yl)urea (MCK139);
1-(3-chlorophenyl)-3-(6-nitrobenzo[d]thiazol-2-yl)urea (MCK140);
1-(4-bromophenyl)-3-(6-nitrobenzo[d]thiazol-2-yl)urea (MCK147);
1-(6-aminobenzo[d]thiazol-2-yl)-3-(3-chlorophenyl)urea (MCK148);
N-(2-(3-(3-chlorophenyl)ureido)benzo[d]thiazol-6-yl)acetamide (MCK149);
N-(2-(3-(3-chlorophenyl)ureido)benzo[d]thiazol-6-yl)methanesulfonamide (MCK150);
1-(3,5-dichlorophenyl)-3-(6-nitrobenzo[d]thiazol-2-yl)urea (MCK151);
1-(4-methoxyphenyl)-3-(6-nitrobenzo[d]thiazol-2-yl)urea (MCK152);
1-(6-nitrobenzo[d]oxazol-2-yl)-3-(m-tolyl)urea (MCK153);
1-(5-nitrobenzo[d]oxazol-2-yl)-3-phenylurea (MCK154);
1-(6-nitro-1H-benzo[d]thiazol-2-yl)-3-phenylurea (MCK155);
1-(3-chlorophenyl)-3-(6-nitro-1 H-benzo[d]imidazol-2-yl)urea (MCK156);
1-(6-nitro-1H-benzo[d]imidazol-2-yl)-3-phenylurea (MCK157);
1-(3-chlorophenyl)-3-(5-nitrobenzo[d]oxazol-2-yl)urea (MCK158);
1-(6-bromobenzo[d]thiazol-2-yl)-3-(3-chlorophenyl)urea (MCK159);
1-(3-chlorophenyl)-3-(6-nitrobenzo[d]oxazol-2-yl)urea (MCK160);
1 1-(3-chlorophenyl)-3-(6-(thiophen-2-yl)benzo[d]thiazol-2-yl)urea (MCK161);
1-(3-chlorophenyl)-3-(6-(furan-2-yl)benzo[d]thiazol-2-yl)urea (MCK162);
1-(3-chlorophenyl)-3-(6-(thiophen-3-yl)benzo[d]thiazol-2-yl)urea (MCK163);
1-(3-chlorophenyl)-3-(6-(naphthalen-1-yl)benzo[d]thiazol-2-yl)urea (MCK164);
1-(2-bromophenyl)-3-(6-nitrobenzo[d]thiazol-2-yl)urea (MCK165);
1-(3-chlorophenyl)-3-(6-ethoxybenzo[d]thiazol-2-yl)urea (MCK166);
1-(3,5-dichlorophenyl)-3-(6-ethoxybenzo[d]thiazol-2-yl)urea (MCK167);
1-(6-nitrobenzo[d]thiazol-2-yl)-3-(p-tolyl)urea (MCK168);
1-(4-chlorophenyl)-3-(6-nitrobenzo[d]thiazol-2-yl)urea (MCK169);
1-(3-chlorophenyl)-3-(6-(pyridin-2-yl)benzo[d]thiazol-2-yl)urea (MCK172); and
1-(6-nitrobenzo[d]thiazol-2-yl)-3-(o-tolyl)urea (MCK173).

25. The method according to claim 16, said method comprising administering a pharmaceutical composition to the subject topically, orally, or parenterally.

26. A compound, a pharmaceutically acceptable salt or a tautomer thereof, of formula (I): 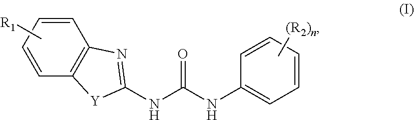
in which:
Y is —S—, or —O—;
R₁ is a radical selected from the group consisting of:
a nitro group, a —NR₃R₄ group with one of R₃ or R₄ is H and the other is a —SO₂R₆ with R₆ is a (C₁-C₆)alkyl group, and
a 3-14 membered ring selected from the group consisting of a thiophenyl, a furanyl, and a naphthalenyl, said 3-14 membered ring is optionally substituted by a radical selected from the group consisting of a (C₁-C₆)alkyl group, a (C₁-C₆)alkyloxy group, a halogen atom, a nitro group, and a carboxyl group;
with the proviso that when R₁ is a nitro group, then Y is —O—;
R₂ is a radical selected from the group consisting of:
a hydrogen atom,
a halogen atom,
a (C₁-C₆)alkyloxy group, and
a (C₁-C₆)alkyl group; and
n is an integer number from 0 to 5.

27. The compound according to claim 26, wherein said compound is selected from the group consisting of:
N-(2-(3-(3-chlorophenyl)ureido)benzo[d]thiazol-6-yl)methanesulfonamide (MCK150);
1-(5-nitrobenzo[d]oxazol-2-yl)-3-phenylurea (MCK154);
1-(3-chlorophenyl)-3-(5-nitrobenzo[d]oxazol-2-yl)urea (MCK158);
1-(3-chlorophenyl)-3-(6-nitrobenzo[d]oxazol-2-yl)urea (MCK160);
1-(3-chlorophenyl)-3-(6-(thiophen-2-yl)benzo[d]thiazol-2-yl)urea (MCK161);
1-(3-chlorophenyl)-3-(6-(furan-2-yl)benzo[d]thiazol-2-yl)urea (MCK162);
1-(3-chlorophenyl)-3-(6-(thiophen-3-yl)benzo[d]thiazol-2-yl)urea (MCK163); and
1-(3-chlorophenyl)-3-(6-(naphthalen-1-yl)benzo[d]thiazol-2-yl)urea (MCK164).

28. A compound selected from the group consisting of:
N-(2-(3-(3-chlorophenyl)ureido)benzo[d]thiazol-6-yl)acetamide (MCK149);
N-(2-(3-(3-chlorophenyl)ureido)benzo[d]thiazol-6-yl)methanesulfonamide (MCK150);
1-(5-nitrobenzo[d]oxazol-2-yl)-3-phenylurea (MCK154);
1-(6-nitro-1H-benzo[d]imidazol-2-yl)-3-phenylurea (MCK157);
1-(3-chlorophenyl)-3-(5-nitrobenzo[d]oxazol-2-yl)urea (MCK158);
1-(3-chlorophenyl)-3-(6-nitrobenzo[d]oxazol-2-yl)urea (MCK160);
1-(3-chlorophenyl)-3-(6-(thiophen-2-yl)benzo[d]thiazol-2-yl)urea (MCK161);
1-(3-chlorophenyl)-3-(6-(furan-2-yl)benzo[d]thiazol-2-yl)urea (MCK162);
1-(3-chlorophenyl)-3-(6-(thiophen-3-yl)benzo[d]thiazol-2-yl)urea (MCK163);
1-(3-chlorophenyl)-3-(6-(naphthalen-1-yl)benzo[d]thiazol-2-yl)urea (MCK164);
1-(3,5-dichlorophenyl)-3-(6-ethoxybenzo[d]thiazol-2-yl)urea (MCK167); and
1-(3-chlorophenyl)-3-(6-(pyridin-2-yl)benzo[d]thiazol-2-yl)urea (MCK172).

29. A pharmaceutical composition comprising a compound according to claim 26 and a pharmaceutically acceptable carrier.

30. A pharmaceutical composition comprising a compound according to claim 28 and a pharmaceutically acceptable carrier.

31. A method of treating cancer comprising administering a compound according to claim 26, or a pharmaceutical composition comprising said compound to a subject having cancer.

32. The method according to claim 31, wherein said compound is selected from the group consisting of:
N-(2-(3-(3-chlorophenyl)ureido)benzo[d]thiazol-6-yl)methanesulfonamide (MCK150);
1-(5-nitrobenzo[d]oxazol-2-yl)-3-phenylurea (MCK154);
1-(3-chlorophenyl)-3-(5-nitrobenzo[d]oxazol-2-yl)urea (MCK158);
1-(3-chlorophenyl)-3-(6-nitrobenzo[d]oxazol-2-yl)urea (MCK160);
1-(3-chlorophenyl)-3-(6-(thiophen-2-yl)benzo[d]thiazol-2-yl)urea (MCK161);
1-(3-chlorophenyl)-3-(6-(furan-2-yl)benzo[d]thiazol-2-yl)urea (MCK162);
1-(3-chlorophenyl)-3-(6-(thiophen-3-yl)benzo[d]thiazol-2-yl)urea (MCK163); and
1-(3-chlorophenyl)-3-(6-(naphthalen-1-yl)benzo[d]thiazol-2-yl)urea (MCK164).

33. A method of treating cancer comprising administering a compound according to claim 28, or a pharmaceutical composition comprising said compound to a subject having cancer.
